(19) United States
(12) Patent Application Publication
(10) Pub. No.: US 2020/0270628 A1
(43) Pub. Date: Aug. 27, 2020

(54) Broad Resistance to Soybean Cyst Nematode

(71) Applicants: Board of Trustees of Southern Illinois University, Columbia, MO (US); The Curators of the University of Missouri, Carbondale, IL (US)

(72) Inventors: Khalid Meksem, Carbondale, IL (US); Henry Nguyen, Columbia, MO (US); Naoufal Lakhssassi, Carbondale, IL (US); Mariola Klepadlo, Columbia, MO (US); Gunvant Patil, St. Paul, MN (US)

(73) Assignees: Board of Trustees of Southern Illinois University, Carbondale, IL (US); The Curators of the University of Missouri, Columbia, MO (US)

(21) Appl. No.: 16/739,985

(22) Filed: Jan. 10, 2020

Related U.S. Application Data
(60) Provisional application No. 62/791,637, filed on Jan. 11, 2019.

(51) Int. Cl. C12N 15/82

(52) U.S. Cl. CPC C12N 15/8285

(57) ABSTRACT

A transgenic soybean plant resistant to soybean cyst nematode (SCN), or parts thereof, are provided. Also provided are methods of increasing SCN resistance of a soybean plant and associated DNA constructs.

### REFERENCE TO RELATED APPLICATIONS

This application claims the benefit of U.S. Provisional Application Ser. No. 62/791,637, filed Jan. 11, 2019, the entire disclosure of which is incorporated herein by reference.

### STATEMENT REGARDING FEDERALLY SPONSORED RESEARCH OR DEVELOPMENT

This invention was made with government support under grant number S1066 awarded by the United States Department of Agriculutre, National Institute of Food and Agriculture. The government has certain rights in the invention.

### FIELD OF THE DISCLOSURE

The present disclosure generally relates to methods of conferring resistance to nematodes in soybeans.

BACKGROUND OF THE DISCLOSURE

Soybean cyst nematode (SCN, Heterodera glycines Ichinohe) is the most devastating pest among plant-parasitic nematode species in the United States and worldwide. Annual soybean yield losses caused by this pest in the United States alone were estimated at $1.5 billion [Wrather & Koenning]. The deployment of SCN resistance soybean varieties is the most efficient management manner to control the nematodes damage in soybean production areas. In past decades, many efforts have been made to evaluate the USDA Soybean Germplasm Collection for new sources of resistance to SCN. Over 100 plant introductions (PIs), including common accessions PI 88788, ‘Peking’ (PI 548402), and PI 437654 were identified as resistant to different SCN HG Types [Concibido et al; Arelli et al., 2000; Arelli et al., 1997]. Among these, PI 437654 and PI 567516C were highly resistant to multiple SCN races [Vuong et al.; Wu et al.; Arelli et al., 2009; Brucker et al.].

To date, only two major sources of resistance have been commonly employed in soybean breeding programs, which are derived from soybean lines PI 88788 and ‘Peking’ [Concibido et al.]. PI 88788 has eight copies at the Rhg1 locus and is the primary source used in commercial breeding programs to battle SCN damage. More than 90% of SCN resistant cultivars are derived from this single source. A survey conducted in 2005 [Niblack et al.] showed that 83% of the soybean fields in Illinois were infested with SCN and 70% of these have adapted to PI 88788, resulting in a reduction of the effectiveness when using SCN resistant cultivars as a crop management tool [Niblack et al.]. It is now urgent for soybean growers to have alternative sources of SCN resistance to overcome the selection pressure and the SCN population shifts.

Recent advances in high-throughput genotyping and next-generation sequencing technologies provide researchers with new opportunities to analyze genome structure at a large and a fine scale [Wang et al.; Schmutz et al., 2014]. Re-sequencing of diverse genetic populations is a powerful approach for trait discovery and has been conducted in a variety of organisms, including humans [Telenti et al], animals [Choi et al.; Zhou et al., 2016; Rubin et al.], and several species thereof [Afolitos et al.; Varshney et al., 2017; Lam et al., 2011; Lam et al., 2010; Xu et al.]. Whole genome re-sequencing (WGRS) facilitates the identification of functional variations and provides a comprehensive catalog of genome wide polymorphism in closely related accessions. It also overcomes the limitation of missing data compared to other genotyping technologies [Jackson et al.]. Importantly, the data from WGRS provides a high resolution of the variation within populations, thus enabling marker-assisted breeding, gene mapping, and the identification of phenotype-genotype relationships. In humans, WGRS of diverse human populations aided the development of HapMap and facilitated the identification of common genetic variations [Gibbs et al.]. In crops such as rice [Huang et al.; Yano et al.], tomato [Aflitos et al.], soybean [Lam et al., 2010], chickpea [Varshney et al., 2013], pigeonpea [Varshney et al., 2017] and maize [Gore et al.], the detailed analysis of re-sequencing data provided a catalog of genetic variants, such as single nucleotide polymorphisms (SNPs) and copy number variation (CNV), across the genome. Furthermore, this information has been used to identify genomic regions that are expected to play an important role during domestication and selection. Importantly, CNVs are an important component of genetic variation because they influence gene expression, phenotypic variation and adaptation by disturbing genes and altering gene dosage [Sebat et al.; Shlien & Malkin; Redon et al.]. In humans, CNVs are associated with cancer risk factors, neurological functions, regulation of cell growth and metabolism [Sebat et al.].

In soybean, a large number of wild accessions, landraces, and varieties have recently been re-sequenced to provide useful information about the genome structure and enable the discovery of new genes [Lam et al., 2010; Zhou et al., 2015; Qi et al.; Schmutz et al., 2010; Li et al.; Valliyodan et al.]. Moreover, the development of soybean high-density markers from large sequencing data sets provides a powerful tool for whole genome prediction and selection applications [Patil et al., 2016]. In the case of SCN resistance, remarkable progress has been made since the cloning of the resistance genes that reside in the two major loci, Rhg 1 and Rhg4 [Liu et al., 2012; Cook et al., 2012; Liu et al., 2017; Lakhssassi et al.]. However, the mechanism of SCN broad-based resistance and the interaction of these two loci in the soybean accessions are still unclear and warrant further investigation.

### SUMMARY OF THE DISCLOSURE

One embodiment of the present disclosure is a transgenic soybean plant resistant to soybean cyst nematode (SCN) comprising a first polynucleotide encoding a serine hydroxymethyltransferase promoter that functions in the soybean plant operably linked to a second polynucleotide encoding a polypeptide having serine hydroxymethyltransferase activity. The first polynucleotide may comprise SEQ ID NO: 1, or a sequence at least 95% identical thereto, or a full-length complement thereof, or a functional fragment thereof. The first polynucleotide may comprise one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-. The transgenic soybean plant may have increased SCN resistance compared to a control soybean plant lacking the first polynucleotide.

Another embodiment of the present disclosure is a transgenic soybean plant resistant to soybean cyst nematode (SCN) comprising a first polynucleotide encoding a serine hydroxymethyltransferase promoter that functions in the soybean plant operably linked to a second polynucleotide encoding a polypeptide having serine hydroxymethyltransferase activity. The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H. The transgenic soybean plant may have increased SCN resistance compared to a control soybean plant lacking the second polynucleotide. The second polynucleotide may have increased expression, an altered expression pattern, or an increased copy number.

Another embodiment of the present disclosure is a plant of an agronomically elite soybean variety comprising a first polynucleotide encoding a serine hydroxymethyltransferase promoter that functions in the soybean plant operably linked to a second polynucleotide encoding a polypeptide having serine hydroxymethyltransferase activity. The first polynucleotide may comprise SEQ ID NO: 1, or a sequence at least 95% identical thereto, or a full-length complement thereof, or a functional fragment thereof. The first polynucleotide may comprise one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-. The plant may have increased soybean cyst nematode (SCN) resistance compared to a control soybean plant lacking the first polynucleotide.

Another embodiment of the present disclosure is a plant of an agronomically elite soybean variety comprising a first polynucleotide encoding a serine hydroxymethyltransferase promoter that functions in the soybean plant operably linked to a second polynucleotide encoding a polypeptide having serine hydroxymethyltransferase activity. The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H. The plant may have increased soybean cyst nematode (SCN) resistance compared to a control soybean plant lacking the second polynucleotide. The second polynucleotide may have increased expression, an altered expression pattern, or an increased copy number.

Another embodiment of the present disclosure is a method of increasing soybean cyst nematode (SCN) resistance of a soybean plant comprising transforming the soybean plant with a first DNA construct comprising a first polynucleotide encoding a serine hydroxymethyltransferase promoter that functions in the soybean plant operably linked to a second polynucleotide encoding a polypeptide having serine hydroxymethyltransferase activity. The first polynucleotide may comprise SEQ ID NO: 1, or a sequence at least 95% identical thereto, or a full-length complement thereof, or a functional fragment thereof. The first polynucleotide may comprise one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-. The transformed soybean plant may have increased SCN resistance compared to a control soybean plant lacking the first polynucleotide.

Another embodiment of the present disclosure is a method of increasing soybean cyst nematode (SCN) resistance of a soybean plant comprising transforming the soybean plant with a first DNA construct comprising a first polynucleotide encoding a serine hydroxymethyltransferase promoter that functions in the soybean plant operably linked to a second polynucleotide encoding a polypeptide having serine hydroxymethyltransferase activity. The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H. The transformed soybean plant may have increased SCN resistance compared to a control soybean plant lacking the second polynucleotide. The second polynucleotide may have increased expression, an altered expression pattern, or an increased copy number.

Another embodiment of the present disclosure is a DNA construct comprising a first polynucleotide encoding a serine hydroxymethyltransferase promoter that functions in a soybean plant operably linked to a second polynucleotide encoding a polypeptide having serine hydroxymethyltransferase activity. The first polynucleotide may comprise SEQ ID NO: 1, or a sequence at least 95% identical thereto, or a full-length complement thereof, or a functional fragment thereof. The first polynucleotide may comprise one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-.

Another embodiment of the present disclosure is a DNA construct comprising a first polynucleotide encoding a serine hydroxymethyltransferase promoter that functions in soybean operably linked to a second polynucleotide encoding a polypeptide having serine hydroxymethyltransferase activity. The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H. The DNA construct may be constructed such that a soybean plant transformed with the DNA construct may have increased expression, an altered expression pattern, or an increased copy number of the second polynucleotide compared to a control soybean plant that has not been transformed with the DNA construct.

### DESCRIPTION OF THE DRAWINGS

The following drawings form part of the present specification and are included to further demonstrate certain aspects of the present disclosure. The present disclosure may be better understood by reference to one or more of these drawings in combination with the detailed description of specific embodiments presented herein. However, those of skill in the art will understand that the drawings, described below, are for illustrative purposes only. The drawings are not intended to limit the scope of the present teachings in any way.

The patent or patent application files contains at least one drawing executed in color. Copies of this patent or patent application publication with color drawing(s) will be provided by the Office upon request and payment of the necessary fee.

FIG. 1A and FIG. 1B is a bar graph showing the female index for SCN Race 1, 2, 3, and 5 from the 106 soybean lines used in the present examples.

FIG. 2A and FIG. 2B is a series of graphs depicting the diversity, linkage disequilibrium (LD) and sequence analysis of region surrounding the Rhg1 and Rhg4 loci.

FIG. 2D is a drawing depicting the diversity, linkage disequilibrium (LD) and a sequence analysis of a region surrounding the Rhg1 and Rhg4 loci.

FIG. 3A and FIG. 3B are drawings illustrating the haplotype clustering, correlation with female index and CNV of the Rhg-1 and Rhg-4 locus in the 106 soybean lines. Schematic graphs show the position of amino acid change (nonsynonymous SNP/indel) for Glyma. 18g022500 (alpha Soluble NSF attachment protein; a-SNAP), and Glyma. 08g108900 (Serine Hydroxymethyl Transferase; SHMT) genes. The SNPs in black background are different to the reference genome (Williams 82). In the gene model diagram (top of the figure), the dark gray box represents exons, the gray bar represents introns, the light gray box represents promoter region, and medium gray box represents 3′ or 5′ UTR. SNPs were positioned relative to the genomic position in the genome version W82.a2. SCN Female Index ratings are shown for each genotype X race combination (races include PA1, PA2, PA3, PAS and PA14).

FIG. 4A is a bar graph depicting copy number variation (CNV) of the Rhg1 locus defined from whole-genome resequencing for SCN-resistant lines.

FIG. 4B is a bar graph depicting copy number variation (CNV) of the Rhg4 locus defined from the whole-genome resequencing for SCN-resistant lines.

FIG. 5 is a table illustrating statistics of DNA variant analysis for Rhg1 from SCN-resistant lines.

FIG. 6 is a table showing the genetic basis of haplotype to haplotype interaction of Rhg1 and Rhg4.

FIG. 7 is a table depicting statistics for DNA variant analysis of the Rhg1 and Rhg4 loci from SCN-resistant lines.

FIG. 8A and FIG. 8B is a graph representing CNV using whole genome sequencing data.

FIG. 9 is a table illustrating comparison and confirmation of the Rhg-1 and Rhg-4 CNV using different platforms from representative SCN-resistant lines.

FIG. 10A and FIG. 10B is a series of graphs showing copy number variation (CNV) of the Rhg1 (A) and Rhg4 (B) loci validated using a comparative genomic hybridization (CGH) method. The color of each spot indicates the relative CNV level at each genomic interval compared to ‘Williams 82’ (which is single copy for both loci). Clear structural differences are exhibited by five out of six tested genotypes at Rhg1 and for three out of six genotypes at Rhg4.

FIG. 11A, FIG. 11B, and FIG. 11C is a drawing depicting homology modeling of the GmSNAP18 and the tetrameric GmSHMT08 from ‘Forrest’ ('Peking'-type resistance). (A) GmSHMT08 tetramer showing the characterized three haplotypes (red) between resistant and susceptible from the 106 soybean lines sequenced. (B) One GmSNAP18 subunit showing the characterized seven haplotypes (yellow) between resistant and susceptible from the 106 soybean lines. Glycine PLP S39, Y59, G132, H134, and R389 residues (Green), Dimerization E35 and E40 residues (Orange), in addition to the folate substrate biding N374 residue (Pink) are shown. (C) The effect on spontaneous occurring mutations on the three haplotypes I37F, R130P, and Y358N/H were mapped into the predicted model.

FIG. 12A and FIG. 12B is a drawing illustrating PCR amplification of the regions surrounding Glyma.08g108900 (Rhg4) in different soybean lines. (A) Graphical illustrations of the regions to be amplified by PCR. (B) Agarose gel images of the amplified PCR products in different soybean lines. The size and location of the repeat was estimated using the sequencing data (>20-kb around SHMT). It was reasoned that if two primers are located inside the repeat, a PCR product of the expected size defined by the primers should be generated. The results suggest that the repeat appears to be longer than 24.8-kb. M-DNA/HindIII size marker.

FIG. 13 is a table listing the primers used to study the Rhg4 duplication.

FIG. 14 is a drawing illustrating the strategies employed to obtain the junction regions between two neighboring repeats. The left most column depicts the two outward primers that were designed to amplify the junction between two neighboring tandem repeats Light arrow: 24k-right-forward primer near the right end of the 24-kb region; dark arrow: 24k-left-reverse primer near the left end of the 24-kb region. The middle column depicts Strategies to amplify the junction between two neighboring inverted repeats (back-to-back or head-to-head) if present. The right most column is a graphical illustration to show that there will not be any PCR band if no neighboring repeats are present.

FIG. 15A, FIG. 15B, FIG. 15C and FIG. 15D is a series of gel images representing amplification of the junction regions between two neighboring repeats in Williams 82, ‘Peking’ (HNO19) and PI 437654 (HNO15) soybean lines. (A) Gel image of the PCR bands obtained for the junction between two neighboring tandem repeats. (B) Gel image of the PCR reactions intended to amplify the regions between two neighboring back-to-back inverted repeats if present. (C) Gel image of the PCR reactions intended to amplify the regions between two neighboring head-to-head inverted repeats. Part of the sequence obtained from sequencing the PCR products circled in (A), showing the joining of two sequences from two different regions in the sequenced Williams 82 reference genome, separated by the extra four bps, TGCA (underlined). The sequences from both ‘Peking’ and PI 437654 were the same.

FIG. 16A and FIG. 16B is a gel image depicting confirmation of the junction regions between two neighboring repeats in different soybean lines. (A) PCR amplification of the junction regions from different soybean lines based on the information obtained in Figure lx. The expected size of the bands was 819 bps. (B) Part of the sequence obtained from sequencing the PCR bands in (A). All the PCR bands from the three lines produced the same junction sequence, which was also the same as presented in FIG. 12.

FIGS. 17A and 17B is a drawing showing the identified repeat at the Rhg4 locus. (A) Illustration of the two neighboring tandem repeats, separated by TGCA (underlined and bolded). Each repeat is 35,705 bps based on the reference genome. (B) Screen shot of the repeat region from the reference genome, together with the genes present in this region.

FIG. 18A and FIG. 18B is a series of tables showing a summary of haplotype clusters, reaction to SCN races, CNV and type of Rhg-1 and Rhg-4 resistance lines. (A) PI88788 and Cloud type resistance. (B) Peking type resistance.

FIG. 19A, FIG. 19B, FIG. 19C, FIG. 19D, FIG. 19E, FIG. 19F, and FIG. 19G is a series of drawings depicting haplotype clustering of GmSHMT08 promoter. (A-F) Schematic graph showing correlation with female index and amino acid changes of the GmSHMT08 and GmSNAP18 protein in 106 soybean lines. (G) Schematic graph showing a subset of beneficial SNPs in the promoter region in a selection of the 106 soybean lines tested. SNP in black background are different to the reference genome (Williams 82).

FIG. 20A1, FIG. 20A2, and FIG. 20B is a series of drawings depicting haplotype clustering of GmSNAP18 promoter. (A) Schematic graph showing correlation with female index and amino acid changes of the GmSHMT08 and GmSHAP18 protein in 106 soybean lines. (B) Schematic graph showing a subset of beneficial SNPs in the promoter region in a selection of the 106 soybean lines tested. SNP in black background are different to the reference genome (Williams 82). SNPs were positioned relative to the genomic position in W82.a2. SCN Female Index rating system: FI=0-9, resistant (moderately dotted shading); 10-29 moderate resistance (boxed shading); 30-59 moderate susceptibility (lightest dotted shading); >60, susceptible (no shading).

FIG. 21 is a drawing illustrating the schematic overview of allelic variants (promoter, amino acid change, CNV) in GmSHMT08 and GmSNAP18 genes and their impact of SCN resistance in five races. SCN Female Index rating system: FI=0-9, resistant (moderately dotted shading); 10-29 moderate resistance (heaviest dotted shading); 30-59 moderate susceptibility (lightest dotted shading); >60, susceptible (no shading). Black and white checked box represents promoter region; black box with white squares represents coding region and vertical lines represents amino acid change. (Not drawn to the scale).

FIG. 22 is a table depicting the requirement of Rhg1 and Rgh4 copies in presence and absence of GmSHMT08 promoter to confer SCN resistance.

FIG. 23 is a table illustrating the female indexes of soybean accessions used for gene expression analysis against five soybean cyst nematode populations: Race 1 (HG Type 2.5.7), Race 2 (HG Type 1.2.5.7), Race 3 (HG Type 0), Race 5 (HG Type 2.5.7), and Race 14 (HG Type 1.3.6.7). *SCN Female Index rating system: FI=0-9, resistant; 10-29, moderate resistance; 30-59 moderate susceptibility; >60, susceptibility.

FIG. 24A and FIG. 24B is a series of bar graphs depicting quantitative RT-PCR analyses of GmSNAP18 and GmSHMT08 in the roots at 2 days in the absence (A) and the presence (B) of SCN infection. (A) Roots at 2 days without SCN infection were used as control. (B) Three SCN races were used (PA3, PAS, and PA14). Six indicator lines representing the CNV and haplotype combinations at the promoter and amino acid sequence of the predicted GmSNAP18 and GmSHMT08 were selected. These lines include ‘Peking’, PI 437654, PI 090763, and PI 88788 lines that carry the resistant GmSHMT08 and GmSNAP18 promoters (all these four lines deemed resistant to SCN). However, ‘Essex’ carries the susceptible GmSHMT08 and GmSNAP18 promoter and is susceptible to SCN. PI 407729 has a different promoter haplotype from both resistant and susceptible lines. Three biological replicates were performed for each line. Numbers on the top of each graph represent the line copy number. The error bar stands for the s.e.m. Asterisks indicate significant differences between samples as determined by ANOVA (****P<0.0001 and **P<0.01).

FIG. 25 is a table illustrating the estimation of CNV using whole genome sequence and comparative genome hybridization in NAM population. The WGRS and CHG data was accessed from Stupar Lab, University of Minnesota, MN.

FIG. 26 is a schematic illustrating the constructs used in the functional analysis performed on the GmSHMT08 promoter carrying the four SNPs at four positions within the 2 Kb promoter.

FIG. 27 is a bar graph showing the cyst number present in tested lines with various GmSHMT08 promoter mutations.

FIG. 28 is a chart showing in silico analysis of the GmSHMT08 promoter.

FIG. 29 is a chart showing MADS SQUAMOSA-box Transcription Factor Binding Sites (TFBS) present at the GmSHMT08 promoter of soybean susceptible lines.

### INCORPORATION OF SEQUENCE LISTING

A sequence listing is being submitted herewith by electronic submission and is hereby incorporated by reference.

SEQ ID NO:1 is a nucleotide sequence for Essex Glyma.08g108900 (Serine Hydroxymetyhltransferase) DNA promoter.

SEQ ID NO:2 is a nucleotide sequence for Essex Glyma.08g108900 (Serine Hydroxymethyltransferase) protein.

SEQ ID NO:3 is a nucleotide sequence for Williams 82 Glyma.18g022500 (alpha Soluble NSF attachment protein) DNA promoter.

SEQ ID NO:4 is a nucleotide sequence for Essex Glyma.18g022500 (alpha Soluble NSF attachment protein) protein.

### DETAILED DESCRIPTION OF THE DISCLOSURE

### Transgenic Soybean Plants

One embodiment of the present disclosure is a transgenic soybean plant resistant to soybean cyst nematode (SCN) comprising a first polynucleotide encoding a serine hydroxymethyltransferase promoter that functions in the soybean plant operably linked to a second polynucleotide encoding a polypeptide having serine hydroxymethyltransferase activity.

The first polynucleotide may comprise SEQ ID NO: 1, or a sequence at least 95% identical thereto, or a full-length complement thereof, or a functional fragment thereof. The first polynucleotide may comprise one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-.

The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H.

The second polynucleotide may have increased expression, an altered expression pattern, or an increased copy number. The second polynucleotide may have a copy number of at least 2. Alternatively, the second polynucleotide may have a copy number of at least 3, at least 4, at least 5, at least 6, at least 7, at least 8, at least 9, at least 10, at least 11, at least 12, at least 13, at least 14, or at least 15.

The transgenic soybean plant may also comprise a third polynucleotide encoding an alpha soluble NSF attachment protein promoter that functions in the soybean plant operably linked to a fourth polynucleotide encoding a polypeptide having alpha soluble NSF attachment protein activity.

The third polynucleotide may comprise SEQ ID NO: 3, or a sequence at least 95% identical thereto, or a full-length complement thereof, or a functional fragment thereof. The third polynucleotide may comprise one or more mutations of SEQ ID NO: 3 selected from the group consisting of: C1161A, C1082A, C1044A, C1025T, A1016C, T997A, C970A, C970-, G829T, G825T, A815C, A363T, T336C, G334A, T328C, T327A, C267G, T157G, T83A, C57T, and T36A.

The polypeptide having alpha soluble NSF attachment protein activity may comprise SEQ ID NO: 4, or a sequence at least 95% identical thereto, or a full-length complement thereof, or a functional fragment thereof. The polypeptide having alpha soluble NSF attachment protein activity may comprise one or more mutations of SEQ ID NO: 4 selected from the group consisting of: A111D, Q203K, D208E, I238V, E285Q, D286Y, D286H, D287E, +287A, +287V, L288I, and +288T.

The fourth polynucleotide may have increased expression, an altered expression pattern, or an increased copy number. The fourth polynucleotide may have a copy number of at least 2. Alternatively, the fourth polynucleotide may have a copy number of at least 3, at least 4, at least 5, at least 6, at least 7, at least 8, at least 9, at least 10, at least 11, at least 12, at least 13, at least 14, or at least 15.

The transgenic soybean plant may have a grain yield of at least about 90%, at least about 94%, at least about 98%, at least about 100%, at least about 105%, or at least about 110% as compared to a control soybean plant lacking the first polynucleotide. For example, the grain yield can be from about 90% to about 110%, from about 94% to about 110%, from about 100% to about 110%, or from about 105% to about 110% as compared to a control soybean plant lacking the first polynucleotide.

The transgenic soybean plant may have increased SCN resistance compared to the control soybean plant lacking the first polynucleotide.

The increased SCN resistance may comprise at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, or at least about 1000% decrease in susceptibility to SCN as compared to the control soybean plant lacking the first polynucleotide.

The increased SCN resistance may comprise a decrease in susceptibility to at least 2 SCN races as compared to the control soybean plant lacking the first polynucleotide. Alternatively, the increased SCN resistance may comprise a decrease in susceptibility to at least 3 SCN races, at least 4 SCN races, at least 5 SCN races, at least 6 SCN races, at least 7 SCN races, at least 8 SCN races, at least 9 SCN races, or at least 10 SCN races as compared to the control soybean plant lacking the first polynucleotide.

Another embodiment of the present disclosure is a transgenic soybean plant resistant to soybean cyst nematode (SCN) comprising a first polynucleotide encoding a serine hydroxymethyltransferase promoter that functions in the soybean plant operably linked to a second polynucleotide encoding a polypeptide having serine hydroxymethyltransferase activity.

The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity ay comprise one or more mutations of SEQ ID NO: 2 selected from the group consisting of: 1107F, P200R, P200-, N459Y, and N459H.

The second polynucleotide may have increased expression, an altered expression pattern, or an increased copy number. The second polynucleotide may have a copy number of at least 2. Alternatively, the second polynucleotide may have a copy number of at least 3, at least 4, at least 5, at least 6, at least 7, at least 8, at least 9, at least 10, at least 11, at least 12, at least 13, at least 14, or at least 15.

The transgenic soybean plant may also comprise a third polynucleotide encoding an alpha soluble NSF attachment protein promoter that functions in soybean operably linked to a fourth polynucleotide encoding a polypeptide having alpha soluble NSF attachment protein activity.

The third polynucleotide may comprise SEQ ID NO: 3, or a sequence at least 95% identical thereto, or a full-length complement thereof, or a functional fragment thereof. The third polynucleotide may comprise one or more mutations of SEQ ID NO: 3 selected from the group consisting of: C1161A, C1082A, C1044A, C1025T, A1016C, T997A, C970A, C970-, G829T, G825T, A815C, A363T, T336C, G334A, T328C, T327A, C267G, T157G, T83A, C57T, and T36A.

The polypeptide having alpha soluble NSF attachment protein activity may comprise SEQ ID NO: 4, or a sequence at least 95% identical thereto, or a full-length complement thereof, or a functional fragment thereof. The polypeptide having alpha soluble NSF attachment protein activity may comprise one or more mutations of SEQ ID NO: 4 selected from the group consisting of: A111D, Q203K, D208E, I238V, E285Q, D286Y, D286H, D287E, +287A, +287V, L2881, and +288T.

The fourth polynucleotide may have increased expression, an altered expression pattern, or an increased copy number. The fourth polynucleotide may have a copy number of at least 2. Alternatively, the fourth polynucleotide may have a copy number of at least 3, at least 4, at least 5, at least 6, at least 7, at least 8, at least 9, at least 10, at least 11, at least 12, at least 13, at least 14, or at least 15.

The transgenic soybean plant may have a grain yield of at least about 90%, at least about 94%, at least about 98%, at least about 100%, at least about 105%, or at least about 110% as compared to a control soybean plant lacking the second polynucleotide. For example, the grain yield can be from about 90% to about 110%, from about 94% to about 110%, from about 100% to about 110%, or from about 105% to about 110% as compared to a control soybean plant lacking the first polynucleotide.

The transgenic soybean plant may have increased SCN resistance compared to the control soybean plant lacking the second polynucleotide.

The increased SCN resistance may comprise at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, or at least about 1000% decrease in susceptibility to SCN as compared to the control soybean plant lacking the second polynucleotide.

The increased SCN resistance may comprise a decrease in susceptibility to at least 2 SCN races as compared to the control soybean plant lacking the second polynucleotide. Alternatively, the increased SCN resistance may comprise a decrease in susceptibility to at least 3 SCN races, at least 4 SCN races, at least 5 SCN races, at least 6 SCN races, at least 7 SCN races, at least 8 SCN races, at least 9 SCN races, or at least 10 SCN races as compared to the control soybean plant lacking the second polynucleotide.

A further embodiment of the disclosed technology is a plant part of any of the transgenic soybean plants described above.

### Agronomically Elite Soybean Varieties

Another embodiment of the present disclosure is a plant of an agronomically elite soybean variety comprising a first polynucleotide encoding a serine hydroxymethyltransferase promoter that functions in the soybean plant operably linked to a second polynucleotide encoding a polypeptide having serine hydroxymethyltransferase activity.

The first polynucleotide may comprise SEQ ID NO: 1, or a sequence at least 95% identical thereto, or a full-length complement thereof, or a functional fragment thereof. The first polynucleotide may comprise one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-.

The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H.

The second polynucleotide may have increased expression, an altered expression pattern, or an increased copy number. The second polynucleotide may have a copy number of at least 2. Alternatively, the second polynucleotide may have a copy number of at least 3, at least 4, at least 5, at least 6, at least 7, at least 8, at least 9, at least 10, at least 11, at least 12, at least 13, at least 14, or at least 15.

The plant may also comprise a third polynucleotide encoding an alpha soluble NSF attachment protein promoter that functions in the soybean plant operably linked to a fourth polynucleotide encoding a polypeptide having alpha soluble NSF attachment protein activity.

The third polynucleotide may comprise SEQ ID NO: 3, or a sequence at least 95% identical thereto, or a full-length complement thereof, or a functional fragment thereof. The third polynucleotide may comprise one or more mutations of SEQ ID NO: 3 selected from the group consisting of: C1161A, C1082A, C1044A, C1025T, A1016C, T997A, C970A, C970-, G829T, G825T, A815C, A363T, T336C, G334A, T328C, T327A, C267G, T157G, T83A, C57T, and T36A.

The polypeptide having alpha soluble NSF attachment protein activity may comprise SEQ ID NO: 4, or a sequence at least 95% identical thereto, or a full-length complement thereof, or a functional fragment thereof. The polypeptide having alpha soluble NSF attachment protein activity may comprise one or more mutations of SEQ ID NO: 4 selected from the group consisting of: A111D, Q203K, D208E, I238V, E285Q, D286Y, D286H, D287E, +287A, +287V, L288I, and +288T.

The fourth polynucleotide may have increased expression, an altered expression pattern, or an increased copy number. The fourth polynucleotide may have a copy number of at least 2. Alternatively, the fourth polynucleotide may have a copy number of at least 3, at least 4, at least 5, at least 6, at least 7, at least 8, at least 9, at least 10, at least 11, at least 12, at least 13, at least 14, or at least 15.

The plant may have a grain yield of at least about 90%, at least about 94%, at least about 98%, at least about 100%, at least about 105%, or at least about 110% as compared to a control soybean plant lacking the first polynucleotide. For example, the grain yield can be from about 90% to about 110%, from about 94% to about 110%, from about 100% to about 110%, or from about 105% to about 110% as compared to a control soybean plant lacking the first polynucleotide.

The plant may have increased soybean cyst nematode (SCN) resistance compared to the control soybean plant lacking the first polynucleotide.

The increased SCN resistance may comprise at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, or at least about 1000% decrease in susceptibility to SCN as compared to the control soybean plant lacking the first polynucleotide.

The increased SCN resistance may comprise a decrease in susceptibility to at least 2 SCN races as compared to the control soybean plant lacking the first polynucleotide. Alternatively, the increased SCN resistance may comprise a decrease in susceptibility to at least 3 SCN races, at least 4 SCN races, at least 5 SCN races, at least 6 SCN races, at least 7 SCN races, at least 8 SCN races, at least 9 SCN races, or at least 10 SCN races as compared to the control soybean plant lacking the first polynucleotide.

Another embodiment of the present disclosure is a plant of an agronomically elite soybean variety, comprising a first polynucleotide encoding a serine hydroxymethyltransferase promoter that functions in the soybean plant operably linked to a second polynucleotide encoding a polypeptide having serine hydroxymethyltransferase activity.

The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H.

The second polynucleotide may have increased expression, an altered expression pattern, or an increased copy number. The second polynucleotide may have a copy number of at least 2. Alternatively, the second polynucleotide may have a copy number of at least 3, at least 4, at least 5, at least 6, at least 7, at least 8, at least 9, at least 10 at least 11, at least 12, at least 13, at least 14, or at least 15.

The plant may also comprise a third polynucleotide encoding an alpha soluble NSF attachment protein promoter that functions in soybean operably linked to a fourth polynucleotide encoding a polypeptide having alpha soluble NSF attachment protein activity.

The third polynucleotide may comprise SEQ ID NO: 3, or a sequence at least 95% identical thereto, or a full-length complement thereof, or a functional fragment thereof. The third polynucleotide may comprise one or more mutations of SEQ ID NO: 3 selected from the group consisting of: C1161A, C1082A, C1044A, C1025T, A1016C, T997A, C970A, C970-, G829T, G825T, A815C, A363T, T336C, G334A, T328C, T327A, C267G, T157G, T83A, C57T, and T36A.

The polypeptide having alpha soluble NSF attachment protein activity may comprise SEQ ID NO: 4, or a sequence at least 95% identical thereto, or a full-length complement thereof, or a functional fragment thereof. The polypeptide having alpha soluble NSH attachment protein activity may comprise one or more mutations of SEQ ID NO: 4 selected from the group consisting of: A111D, Q203K, D208E, I238V, E285Q, D286Y, D286H, D287E, +287A, +287V, L2881, and +288T.

The fourth polynucleotide may have increased expression, an altered expression pattern, or an increased copy number. The fourth polynucleotide may have a copy number of at least 2. Alternatively, the fourth polynucleotide may have a copy number of at least 3, at least 4, at least 5, at least 6, at least 7, at least 8, at least 9, at least 10, at least 11, at least 12, at least 13, at least 14, or at least 15.

The plant may have a grain yield of at least about 90%, at least about 94%, at least about 98%, at least about 100%, at least about 105%, or at least about 110% as compared to a control soybean plant lacking the second polynucleotide. For example, the grain yield can be from about 90% to about 110%, from about 94% to about 110%, from about 100% to about 110%, or from about 105% to about 110% as compared to a control soybean plant lacking the first polynucleotide.

The plant may have increased soybean cyst nematode (SCN) resistance compared to the control soybean plant lacking the second polynucleotide.

The increased SCN resistance may comprise at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, or at least about 1000% decrease in susceptibility to SCN as compared to the control soybean plant lacking the second polynucleotide.

The increased SCN resistance may comprise a decrease in susceptibility to at least 2 SCN races as compared to the control soybean plant lacking the second polynucleotide. Alternatively, the increased SCN resistance may comprise a decrease in susceptibility to at least 3 SCN races, at least 4 SCN races, at least 5 SCN races, at least 6 SCN races, at least 7 SCN races, at least 8 SCN races, at least 9 SCN races, or at least 10 SCN races as compared to the control soybean plant lacking the second polynucleotide.

A further embodiment of the disclosed technology is a plant part of any of the plants described above.

### Methods of Increasing SCN Resistance

Another embodiment of the present disclosure is a method of increasing soybean cyst nematode (SCN) resistance of a soybean plant comprising transforming the soybean plant with a first DNA construct comprising a first polynucleotide encoding a serine hydroxymethyltransferase promoter that functions in the soybean plant operably linked to a second polynucleotide encoding a polypeptide having serine hydroxymethyltransferase activity.

The first polynucleotide may comprise SEQ ID NO: 1, or a sequence at least 95% identical thereto, or a full-length complement thereof, or a functional fragment thereof. The first polynucleotide may comprise one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-.

The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H.

The second polynucleotide may have increased expression, an altered expression pattern, or an increased copy number. The second polynucleotide may have a copy number of at least 2. Alternatively, the second polynucleotide may have a copy number of at least 3, at least 4, at least 5, at least 6, at least 7, at least 8, at least 9, at least 10, at least 11, at least 12, at least 13, at least 14, or at least 15.

The method may comprise further transforming the soybean plant with a second DNA construct comprising a third polynucleotide encoding an alpha soluble NSF attachment protein promoter that functions in the soybean plant operably linked to a fourth polynucleotide encoding a polypeptide having alpha soluble NSF attachment protein activity.

The third polynucleotide may comprise SEQ ID NO: 3, or a sequence at least 95% identical thereto, or a full-length complement thereof, or a functional fragment thereof. The third polynucleotide may comprise one or more mutations of SEQ ID NO: 3 selected from the group consisting of: C1161A, C1082A, C1044A, C1025T, A1016C, T997A, C970A, C970-, G829T, G825T, A815C, A363T, T336C, G334A, T328C, T327A, C267G, T157G, T83A, C57T, and T36A.

The polypeptide having alpha soluble NSF attachment protein activity may comprise SEQ ID NO: 4, or a sequence at least 95% identical thereto, or a full-length complement thereof, or a functional fragment thereof. The polypeptide having alpha soluble NSF attachment protein activity may comprise one or more mutations of SEQ ID NO: 4 selected from the group consisting of: A111D, Q203K, D208E, I238V, E285Q, D286Y, D286H, D287E, +287A, +287V, L2881, and +288T.

The fourth polynucleotide may have increased expression, an altered expression pattern, or an increased copy number. The fourth polynucleotide may have a copy number of at least 2. Alternatively, the fourth polynucleotide may have a copy number of at least 3, at least 4, at least 5, at least 6, at least 7, at least 8, at least 9, at least 10, at least 11, at least 12, at least 13, at least 14, or at least 15.

The soybean plant may be simultaneously transformed with the first DNA construct and the second DNA construct. The soybean plant may be transformed separately with the first DNA construct and the second DNA construct. The soybean plant may be transformed first with the first DNA construct then transformed with the second DNA construct. The soybean plant may be transformed first with the second DNA construct then transformed with the first DNA construct.

The transformed soybean plant may have a grain yield of at least about 90%, at least about 94%, at least about 98%, at least about 100%, at least about 105%, or at least about 110% as compared to a control soybean plant lacking the first polynucleotide. For example, the grain yield can be from about 90% to about 110%, from about 94% to about 110%, from about 100% to about 110%, or from about 105% to about 110% as compared to a control soybean plant lacking the first polynucleotide.

The transformed soybean plant may have increased SCN resistance compared to the control soybean plant lacking the first polynucleotide.

The increased SCN resistance may comprise at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, or at least about 1000% decrease in susceptibility to SCN as compared to the control soybean plant lacking the first polynucleotide.

The increased SCN resistance may comprise a decrease in susceptibility to at least 2 SCN races as compared to the control soybean plant lacking the first polynucleotide. Alternatively, the increased SCN resistance may comprise a decrease in susceptibility to at least 3 SCN races, at least 4 SCN races, at least 5 SCN races, at least 6 SCN races, at least 7 SCN races, at least 8 SCN races, at least 9 SCN races, or at least 10 SCN races as compared to the control soybean plant lacking the first polynucleotide.

Another embodiment of the present disclosure is a method of increasing soybean cyst nematode (SCN) resistance of a soybean plant comprising transforming the soybean plant with a first DNA construct comprising a first polynucleotide encoding a serine hydroxymethyltransferase promoter that functions in the soybean plant operably linked to a second polynucleotide encoding a polypeptide having serine hydroxymethyltransferase activity.

The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H.

The second polynucleotide may have increased expression, an altered expression pattern, or an increased copy number. The second polynucleotide may have a copy number of at least 2. Alternatively, the second polynucleotide may have a copy number of at least 3, at least 4, at least 5, at least 6, at least 7, at least 8, at least 9, at least 10, at least 11, at least 12, at least 13, at least 14, or at least 15.

The method may comprise further transforming the soybean plant with a second DNA construct comprising a third polynucleotide encoding an alpha soluble NSF attachment protein promoter that functions in the soybean plant operably linked to a fourth polynucleotide encoding a polypeptide having alpha soluble NSF attachment protein activity.

The third polynucleotide may comprise SEQ ID NO: 3, or a sequence at least 95% identical thereto, or a full-length complement thereof, or a functional fragment thereof. The third polynucleotide may comprise one or more mutations of SEQ ID NO: 3 selected from the group consisting of: C1161A, C1082A, C1044A, C1025T, A1016C, T997A, C970A, C970-, G829T, G825T, A815C, A363T, T336C, G334A, T328C, T327A, C267G, T157G, T83A, C57T, and T36A.

The polypeptide having alpha soluble NSF attachment protein activity may comprise SEQ ID NO: 4, or a sequence at least 95% identical thereto, or a full-length complement thereof, or a functional fragment thereof. The polypeptide having alpha soluble NSF attachment protein activity may comprise one or more mutations of SEQ ID NO: 4 selected from the group consisting of: A111D, Q203K, D208E, I238V, E285Q, D286Y, D286H, D287E, +287A, +287V, L288I, and +288T.

The fourth polynucleotide may have increased expression, an altered expression pattern, or an increased copy number. The fourth polynucleotide may have a copy number of at least 2. Alternatively, the fourth polynucleotide may have a copy number of at least 3, at least 4, at least 5, at least 6, at least 7, at least 8, at least 9, at least 10, at least 11, at least 12, at least 13, at least 14, or at least 15.

The soybean plant may be simultaneously transformed with the first DNA construct and the second DNA construct. The soybean plant may be transformed separately with the first DNA construct and the second DNA construct. The soybean plant may be transformed first with the first DNA construct then transformed with the second DNA construct. The soybean plant may be transformed first with the second DNA construct then transformed with the first DNA construct.

The transformed soybean plant may have a grain yield of at least about 90%, at least about 94%, at least about 98%, at least about 100%, at least about 105%, or at least about 110% as compared to a control soybean plant lacking the second polynucleotide. For example, the grain yield can be from about 90% to about 110%, from about 94% to about 110%, from about 100% to about 110%, or from about 105% to about 110% as compared to a control soybean plant lacking the first polynucleotide.

The transformed soybean plant may have increased SCN resistance compared to the control soybean plant lacking the second polynucleotide.

The increased SCN resistance may comprise at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, or at least about 1000% decrease in susceptibility to SCN as compared to the control soybean plant lacking the second polynucleotide.

The increased SCN resistance may comprise a decrease in susceptibility to at least two SCN races as compared to the control soybean plant lacking the second polynucleotide. Alternatively, the increased SCN resistance may comprise a decrease in susceptibility to at least 3 SCN races, at least 4 SCN races, at least 5 SCN races, at least 6 SCN races, at least 7 SCN races, at least 8 SCN races, at least 9 SCN races, or at least 10 SCN races as compared to the control soybean plant lacking the second polynucleotide.

### DNA Constructs

Another embodiment of the present disclosure is a DNA construct comprising a first polynucleotide encoding a serine hydroxymethyltransferase promoter that functions in a soybean plant operably linked to a second polynucleotide encoding a polypeptide having serine hydroxymethyltransferase activity.

The first polynucleotide may comprise SEQ ID NO: 1, or a sequence at least 95% identical thereto, or a full-length complement thereof, or a functional fragment thereof. The first polynucleotide may comprise one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-.

Another embodiment of the present disclosure is a DNA construct comprising a first polynucleotide encoding a serine hydroxymethyltransferase promoter that functions in soybean operably linked to a second polynucleotide encoding a polypeptide having serine hydroxymethyltransferase activity.

The polypeptide having serine hydroxymethyltransferase activity may comprise SEQ ID NO: 2, or a sequence at least 95% identical thereto, or a full-length complement thereof, or a functional fragment thereof. The polypeptide having serine hydroxymethyltransferase activity may comprise one or more mutations of SEQ ID NO: 2 selected from the group consisting of: 1107F, P200R, P200-, N459Y, and N459H.

The DNA construct may be constructed such that a soybean plant transformed with the DNA construct may have increased expression, an altered expression pattern, or an increased copy number of the second polynucleotide compared to a control soybean plant that has not been transformed with the DNA construct.

### Sequences and Mutations

The amino acid sequences and nucleic acid sequences described herein may contain various mutations. Mutations may include insertions, substitutions, and deletions. Insertions are written as follows: (+)(amino acid/nucleic acid sequence position number)(inserted amino acid/nucleic acid base). For example, +287A would mean an insertion of an alanine residue after position 287 in the corresponding amino acid sequence. Substitutions are written as follows: (amino acid/nucleic acid base to be replaced)(amino acid/nucleic acid sequence position number)(substituted amino acid/nucleic acid base). For example, C1082A would mean a substitution of an adenine base instead of a cytosine base at position 1082 in the corresponding nucleic acid sequence. Deletions are written as follows: (amino acid/nucleic acid base to be deleted)(amino acid/nucleic acid sequence position number)(-). For example, C970- would mean a deletion of the cytosine base normally located at position 970 in the corresponding nucleic acid sequence.

The amino acid sequences and nucleic acid sequences described herein may contain mutations at various sequence positions. Sequence positions may be written a variety a ways for convenience. More specifically, sequence positions may be written from either the beginning of the sequence as a positive position number, or from the end of the sequence as a negative number. Sequence positions may be converted easily between a positive notation and a negative notation by comparing to the sequence length and either adding or subtracting the sequence length. For example, a promoter containing 10 nucleic acid bases with a mutation from cytosine to adenine at the second position from the start of the sequence may be written as C2A. Alternatively, this mutation may be written as C(−9)A, −9C/A, or in a similar fashion denoting the negative position number.

### Definitions and Alternate Embodiments

The following definitions and methods are provided to better define the present disclosure and to guide those of ordinary skill in the art in the practice of the present disclosure. Unless otherwise noted, terms are to be understood according to conventional usage by those of ordinary skill in the relevant art.

The term “agronomically elite” refers to a genotype that has a culmination of many distinguishable traits such as emergence, vigor, vegetative vigor, disease resistance, seed set, standability, and threshability, which allows a producer to harvest a product of commercial significance.

An “allele” refers to one of two or more alternative forms of a genomic sequence at a given locus on a chromosome.

The term “chimeric” is understood to refer to the product of the fusion of portions of two or more different polynucleotide molecules. “Chimeric promoter” is understood to refer to a promoter produced through the manipulation of known promoters or other polynucleotide molecules. Such chimeric promoters can combine enhancer domains that can confer or modulate gene expression from one or more promoters or regulatory elements, for example, by fusing a heterologous enhancer domain from a first promoter to a second promoter with its own partial or complete regulatory elements. Thus, the design, construction, and use of chimeric promoters according to the methods disclosed herein for modulating the expression of operably linked polynucleotide sequences are encompassed by the present disclosure.

Novel chimeric promoters can be designed or engineered by a number of methods. For example, a chimeric promoter may be produced by fusing an enhancer domain from a first promoter to a second promoter. The resultant chimeric promoter may have novel expression properties relative to the first or second promoters. Novel chimeric promoters can be constructed such that the enhancer domain from a first promoter is fused at the 5′ end, at the 3′ end, or at any position internal to the second promoter.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule, which can play an important role in translation initiation and can also be a genetic component in an expression construct. These additional upstream and downstream regulatory nucleic acid molecules may be derived from a source that is native or heterologous with respect to the other elements present on the promoter construct.

“Expression vector”, “vector”, “expression construct”, “vector construct”, “plasmid”, or “recombinant DNA construct” is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

The term “genotype” means the specific allelic makeup of a plant.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Highly stringent hybridization conditions” are defined as hybridization at 65° C. in a 6× SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6× SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6(logio[Na^{+]})+0.41(fraction G/C content)−0.63(% formamide)−(600/1). Furthermore, the Tm of a DNA:DNA hybrid is decreased by 1-1.5° C. for every 1% decrease in nucleotide identity (see Sambrook and Russel, 2006).

The term “introgressed,” when used in reference to a genetic locus, refers to a genetic locus that has been introduced into a new genetic background. Introgression of a genetic locus can thus be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

The term “linked,” when used in the context of nucleic acid markers and/or genomic regions, means that the markers and/or genomic regions are located on the same linkage group or chromosome.

A “marker” means a detectable characteristic that can be used to discriminate between organisms. Examples of such characteristics include, but are not limited to, genetic markers, biochemical markers, metabolites, morphological characteristics, and agronomic characteristics.

A “marker gene” refers to any transcribable nucleic acid molecule whose expression can be screened for or scored in some way.

Certain genetic markers useful in the present disclosure include “dominant” or “codominant” markers. “Codominant” markers reveal the presence of two or more alleles (two per diploid individual). “Dominant” markers reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

The term “phenotype” means the detectable characteristics of a cell or organism that can be influenced by gene expression.

The term “plant” can include plant cells, plant protoplasts, plant cells of tissue culture from which a plant can be regenerated, plant calli, plant clumps and plant cells that are intact in plants or parts of plants such as pollen, flowers, seeds, leaves, stems, and the like. Each of these terms can apply to a soybean “plant”. Plant parts (e.g., soybean parts) include, but are not limited to, pollen, an ovule and a cell.

The term “population” means a genetically heterogeneous collection of plants that share a common parental derivation.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the transcription start site, such as, in the case of a polymerase II type promoter, a TATA element. A promoter can optionally include distal enhancer or repressor elements, which can be located as much as several thousand base pairs from the start site of transcription.

A “quantitative trait locus (QTL)” is a chromosomal location that encodes for alleles that affect the expressivity of a phenotype.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (Sambrook and Russel, 2006; Ausubel et al.; Sambrook and Russel, 2001; Elhai and Wolk).

The “transcription start site” or “initiation site” is the position surrounding a nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) can be denominated as negative.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a plant into which a heterologous nucleic acid molecule has been introduced. The nucleic acid molecule can be stably integrated into the genome as generally known in the art. Known methods of polymerase chain reaction (PCR) include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

The terms “variety” and “cultivar” mean a group of similar plants that by their genetic pedigrees and performance can be identified from other varieties within the same species.

“Wild-type” refers to a virus or organism found in nature without any known mutation.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and, if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (available from DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

In some embodiments, the terms “a,” “an,” “the,” and similar references used in the context of describing a particular embodiment (especially in the context of certain claims) can be construed to cover both the singular and the plural, unless specifically noted otherwise. When used in conjunction with the word “comprising” or other open language in the claims, the words “a” and “an” denote “one or more,” unless specifically noted.

In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that all of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and this can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

As described further below, WGRS data from a diverse panel of 106 soybean accessions was utilized, including wild accessions, exotic germplasm, breeding lines, and varieties, to investigate the two major SCN resistance loci using genome data mining approaches. These efforts provide new insight into the interconnectedness of haplotype compatibility, copy number variation (CNV), promoter variation and gene expression with broad-based SCN resistance.

### Example 1

### Plant Materials and SCN Bioassays.

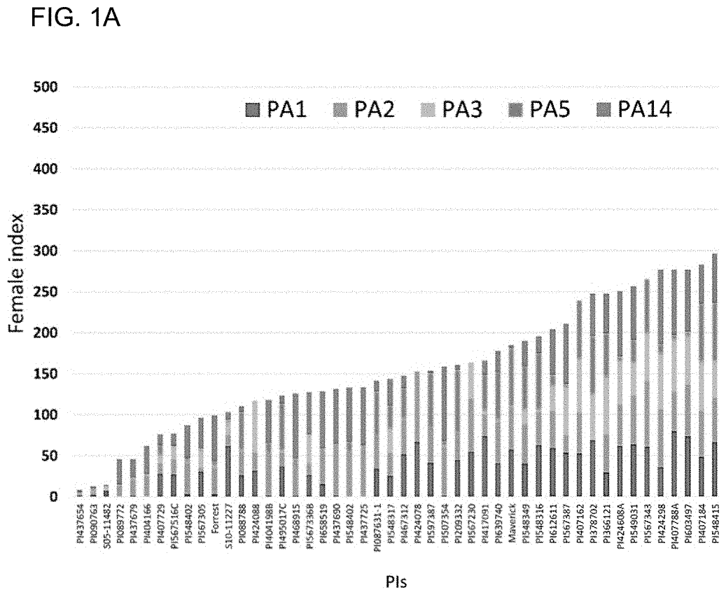

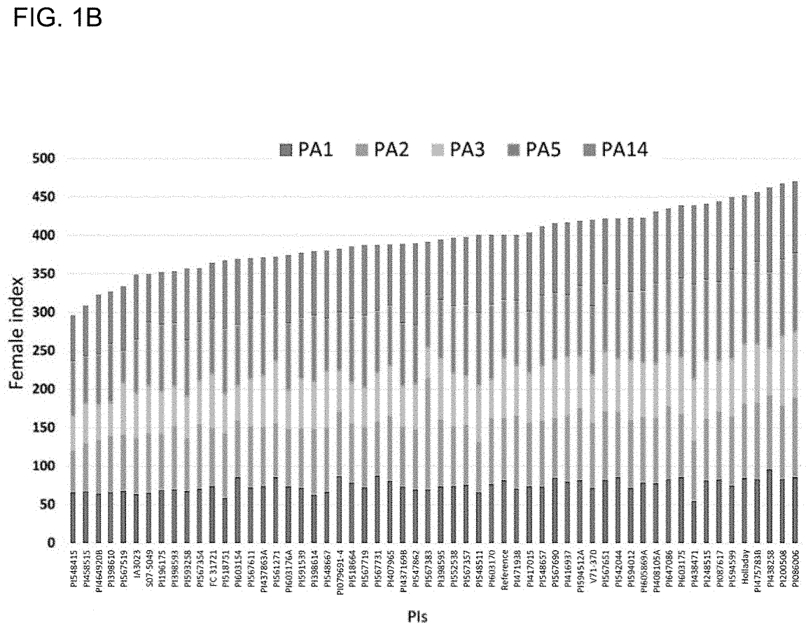

One hundred and six (106) soybean accessions and ‘Forrest’ indicator lines in the present study were evaluated for resistance to different HG Types of SCN. Homogenous nematode populations of races PA1 (HG Type 2.5.7), PA2 (HG Type 1.2.5.7), PA3 (HG Type 0), PA5 (HG Type 2.5.7), and PA14 (HG Type 1.3.5.6.7) have been maintained at the University of Missouri for more than 30 generations. The SCN bioassays were performed in a greenhouse at the University of Missouri following a well-established method [Arelli et al., 1997]. Briefly, soybean seeds were germinated in paper pouches for 3-4 days and were then transplanted into PVC tubes (100 cm³) (one plant per tube). The tubes were filled with steam pasteurized sandy soil and packed into plastic containers prior to transplanting. Each container held 25 tubes and was suspended over water baths maintained at 27±1° C. Five plants of each indicator line were arranged in a randomized complete block design. Two days after transplanting, each plant was inoculated with 2000±25 SCN eggs. Thirty days post inoculation, nematode cysts were washed from the roots of each plant and counted using a fluorescence-based imaging system [Brown et al.]. The female index (FI %) was estimated to evaluate the response of each plant to each race of SCN using the following formula: FI (%)=(average number of female cyst nematodes on a given individual/ average number of female nematodes on the susceptible check)×100. The FI values for all 106 lines are shown in FIG. 1.

### Example 2

### Variant Calling and Haplotype Analysis

The 106 soybean germplasm lines sequenced at approximately 17× genome coverage were utilized for mapping and detection of allelic variants [Valliyodan et al.]. The paired—end resequencing reads were mapped to the soybean reference genome, Williams 82 version 2 (W82.a2.v1.1) with BWA as described previously [Zhou et al., 2015; Valliyodan et al.]. SNP and Indels detection was performed using Genome Analysis Toolkit (GATK, V3.4.0) [McKenna et al.] and SAMTools. For Indel calling, insertions and deletions shorter than or equal to 6 bp were taken into consideration. CNV were detected according to depth distribution of each line [Zhou et al., 2015]. Regions were regarded as CNVs if their minimum length was greater than 2 kb and their mean depth was less than half of the sequence depth or more than double of the sequence depth. The initial and final minimum probability to merge the adjacent breakpoint were set to 0.5 and 0.8, respectively. Additionally, CNV of indicator lines was visualized using GenomeBrowse. Haplotype analysis of the Rhg1 and Rhg4 loci was performed using a pipeline as previously described [Patil et al., 2016]. Briefly, SNP haplotypes were examined by generating map and genotype data files and clustering pictorial output for the Rhgl and Rhg4 genomic regions were visualized using FLAPJACK [Milne et al.]. The SNP identified from each line were clustered based on Neighbor-Joining (NJ) tree output and SNPs were further analyzed for possible synonymous/non-synonymous variation by translation into amino acid sequences. The SNP diversity, average pairwise divergence within population (Ow), Watterson's estimator (θ_{w}), and F_{ST} were estimated as previously described [Valliyodan et al.].

### Example 3

### Comparative Genomic Hybridizations, Taqman Assays, and Digital PCR

Comparative genomic hybridizations (CGH) assay was adapted as described [McHale et al.; Dobbels et al.]. The Taqman assay and digital PCR were performed as previously described [Kadam et al.; Wan et al.]. Briefly, 20 μl reaction was prepared, consisting of 10 μl 2× master reaction mix (Life Technologies, Mass., USA), 1 μl assay mix (18 μM Forward and 18 μM reverse primers+5 μM probe), 1 μl DNA, and 9 μl ddH2O. A 14.5 μl of the PCR mixture was loaded onto a QuantStudio™ 3D Digital PCR 20K Chip. The chip was covered with immersion fluid, a lid was applied, the assembly was filled with immersion fluid, and the loading port was sealed according to the manufacturer's instructions. The chips were loaded into the Dual Flat Block GeneAmpR PCR System 9700 (Life Technologies, Waltham, Mass., USA), and PCR was performed using the following conditions: 96 ° C. for 10 min; 60 ° C. for 2 min and 98 ° C. for 30 seconds, for 39 cycles; 60 ° C. for 2 min; 10 ° C. for storage. The Digital PCR 20K Chip was read using the QuantStudio™ 3D Digital PCR Chip Reader, and the data was analyzed using the QuantStudio™ 3D AnalysisSuiteTM Software (Thermo Fisher Scientific, Waltham, Mass., USA).

### Example 4

### Identification of Tandem Repeats at the Rhg4 Locus

Aliquots of the genomic DNA samples isolated for whole-genome resequencing were used in PCR reactions. The PCR reactions were conducted using PrimeSTAR GXL DNA Polymerase (Takara Bio USA, Inc., formerly known as Clontech Laboratories, Mountain View, Calif., USA), according to the manufacturer's instructions.

### Example 5

### Protein Homology Modeling of GmSNAP18 and GmSHMT08 and Interaction Analysis

Homology modeling of a putative GmSNAP18 and GmSHMT08 protein structure was conducted as previously described [Liu et al., 2017]. To induce and map the corresponding existing natural mutations (haplotypes) between the susceptible and resistant soybeans lines of the GmSHMT08 protein, the structural editing tool from UCSF Chimera package was employed. Additionally, the impact of catalytic activity of the enzyme homodimerization, tetramerization and/or substrate binding was studied. Approximately 5.0 angstroms containing all atoms/bonds of any residue surrounding the mutated residue has been selected first and shown in the model to study all possible residue interactions. Next, the rotamers tool was used to mutate the three residues and study their possible impact on protein activity and/or structure.

### Example 6

### qRT-PCT of GmSNAP18 and GmSHMT08 Genes

Three-day old soybean seedlings of different indicator lines were germinated and inoculated with freshly hatched second-stage juveniles of SCN race PA3, PAS and PA14 as previously described [Rambani et al.]. Three biological samples of inoculated and non-inoculated root tissues were collected at 2 days' post inoculation and used for RNA extraction and qPCR analysis. Total RNA was isolated using Qiagen RNeasy Plant Mini Kit (cat #74904) from root samples collected two days after SCN infection. Total RNA was DNase treated and purified using Turbo DNA-free Kit (QAmbion/Life Technologies AM1907). RNA was quantified using Nanodrop 1000 (V3.7), then a total of 400 nanograms of treated RNA was used to generate cDNA using the cDNA synthesis Kit (Thermoscript, Life Technologies, #11146-025), with random hexamers. About 1/10th of a 20 microliter reverse transcription reaction was used in gene specific qPCR with the Power SYBR® Green PCR Master Mix Kit (Applied Biosystems™ #4368706). Primers used in this study were described previously [Rambani et al.]. For each line, RNA from three biological replicates were used for quantification and then normalized using the deltadelta C_{q} method with Ubiquitin used as a reference gene (ΔCq=C_{q(TAR)}−C_{q(REF)}. Each gene's expression was exponentially transformed to the expression level using the formula (ΔCq Expression=2^{·ΔCq}). Each sample was run in parallel with a control in which RT was not included in the cDNA synthesis reaction.

### Example 7

### Diversity, Disequilibrium and Signatures of Selection at the Rhg1 and Rhg4 Loci

Methods proceeded according to Examples 1-6, unless described otherwise.

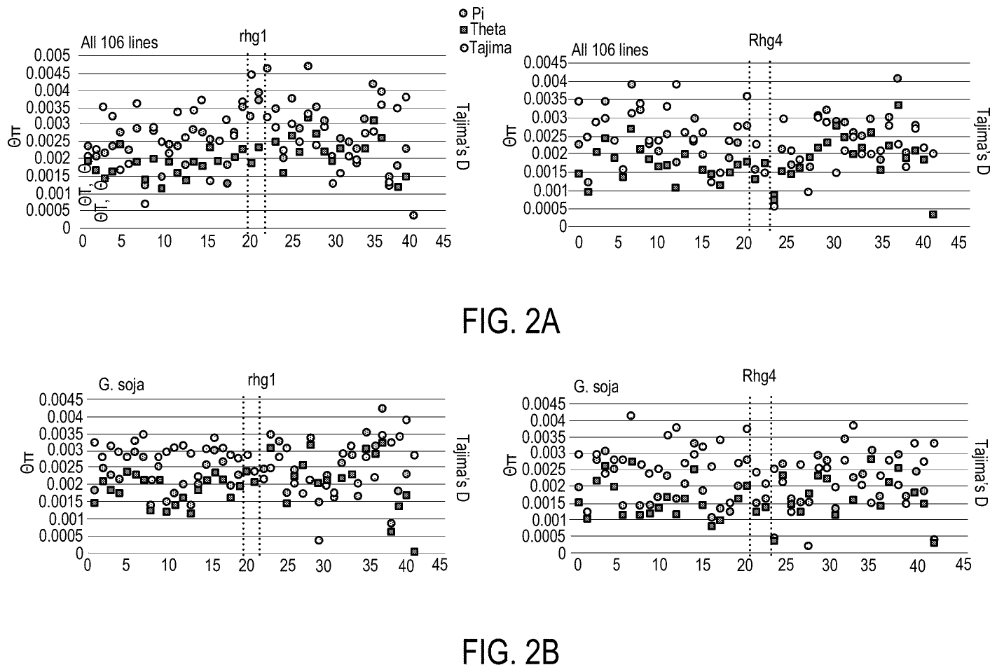

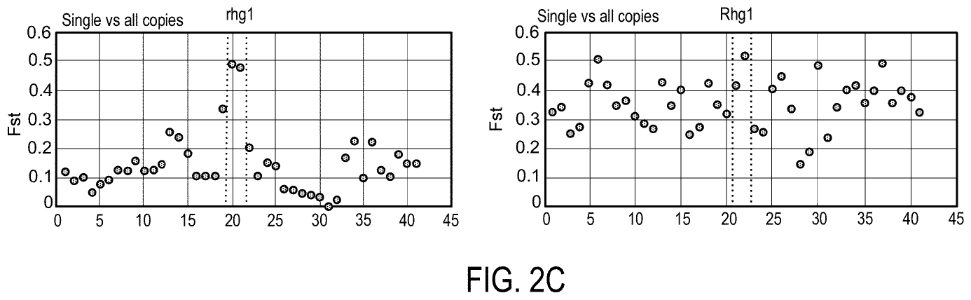

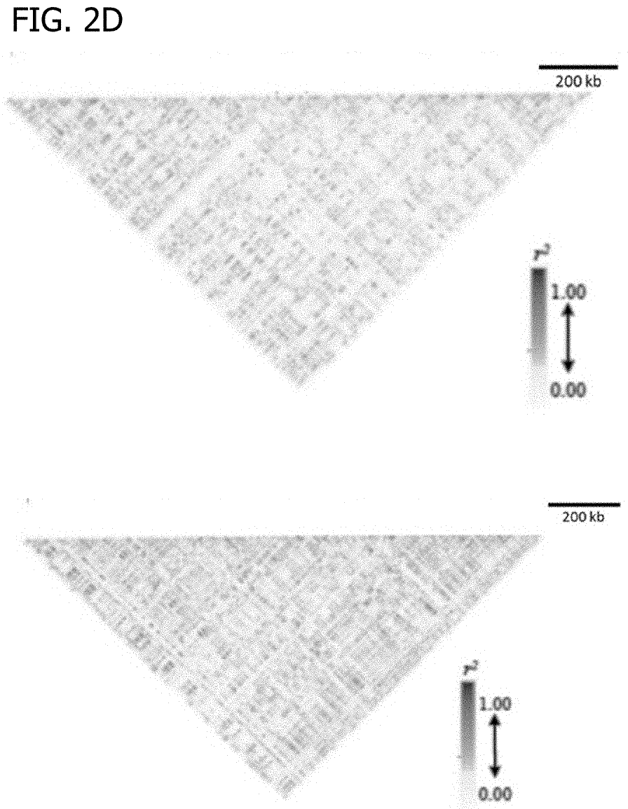

In soybean, the SCN resistance QTL on chromosomes 18 (Rhg1) and 8 (Rhg4) are the two major QTL that have been identified and reported in several publications [Vuong et al.; Liu et al., 2012; Cook et al., 2012]. To investigate the sequence diversity and disequilibrium of the Rhg1 and Rhg4 loci, 1-Mb regions on either side of these loci were analyzed in 106 WGRS lines representing >96% of the sequence diversity [Valliyodan et al.]. The value of θπ, θw, and Tajima's D were estimated for related regions using sliding windows of 50kb extreme allele frequency differentiation over extended linked regions was observed. As the location neared the Rhg1 locus, On increased greatly in the 100-kb region (FIG. 2A). The value of nucleotide diversity at the Rhg1 locus is approximately π=0.00315, which is almost two times greater than the G. max average (0.00178) for all 106 lines. In contrast, a relatively low nucleotide diversity (θπ=0.00159) at the Rhg4 locus was observed (FIG. 2A). Moreover, low nucleotide diversity was observed at both the Rhg1 and Rhg4 loci if only G. soja (7 lines out of 106) was considered for analysis (FIG. 2B), which could be attributed to the fact that SCN resistance is acquired during the domestication process of soybean. A higher Fst value (P<0.005) was also associated with population differentiation near the Rhg1 locus when the multi-copied Rhg1 genotypes were compared with single-copy Rhg-1 genotypes (FIG. 2C). In the case of Rhg4, a relatively similar high Fst value (P<0.01) was observed when the multi-copied Rhg4 genotypes were compared with single-copy Rhg4 genotypes. Linkage disequilibrium (LD) surrounding the Rhg1 and Rhg4 loci was further investigated. The LD (measured by r²) within the ˜200 kb of the Rhg1 and Rhg4 loci was strong and statistically significant, suggesting a block of strong LD extending to ˜100 kb on both sides of the Rhg1 and Rhg4 loci (FIG. 2D).

### Example 8

### Haplotypes Grouping

Methods proceeded according to Examples 1-6, unless described otherwise.

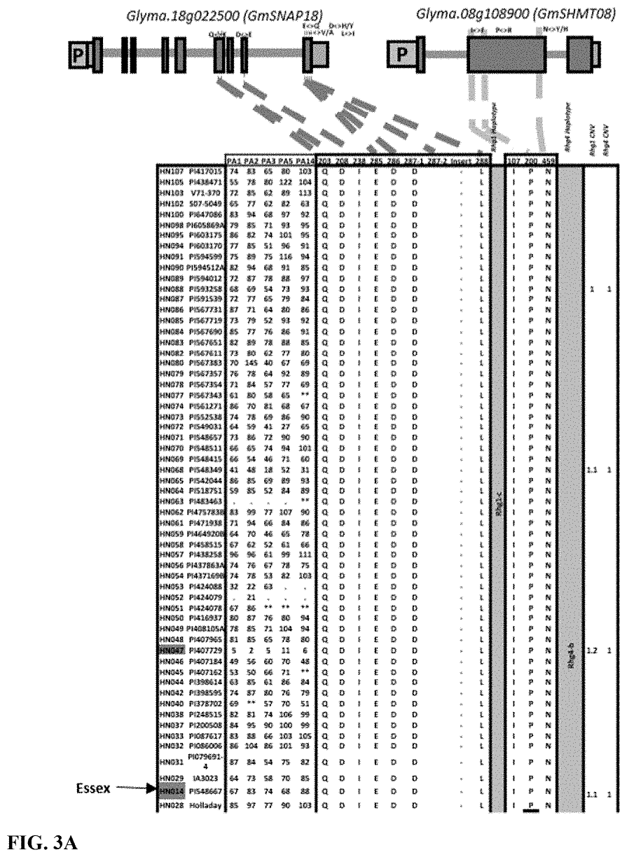

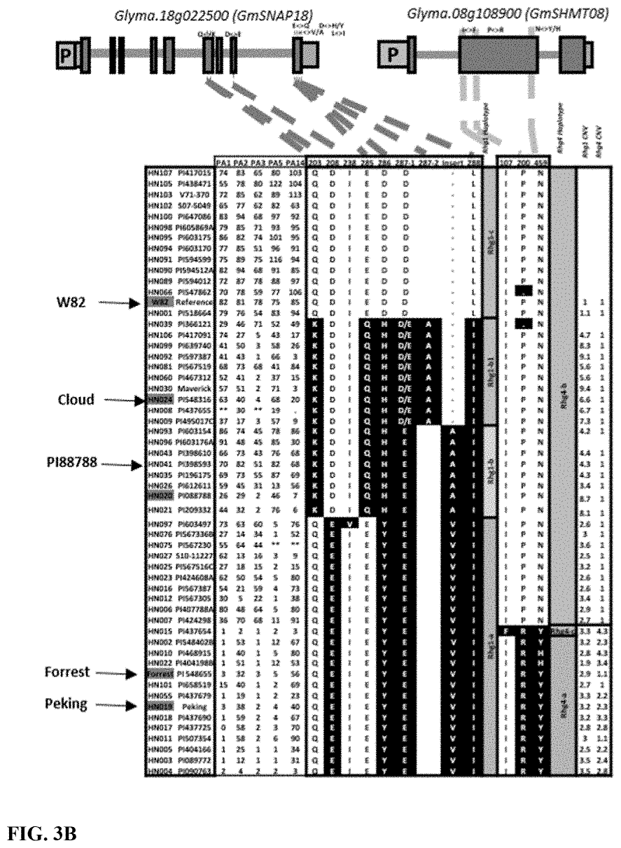

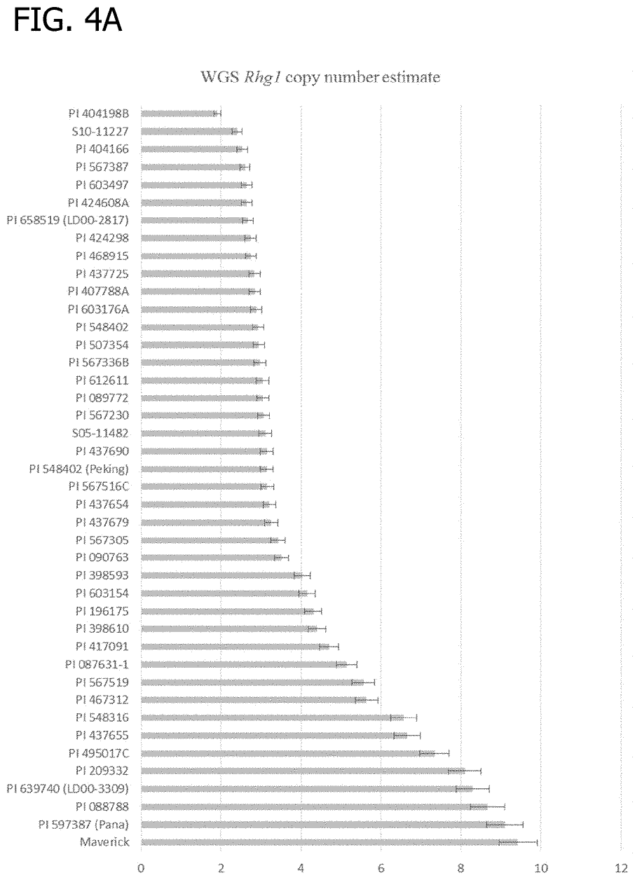

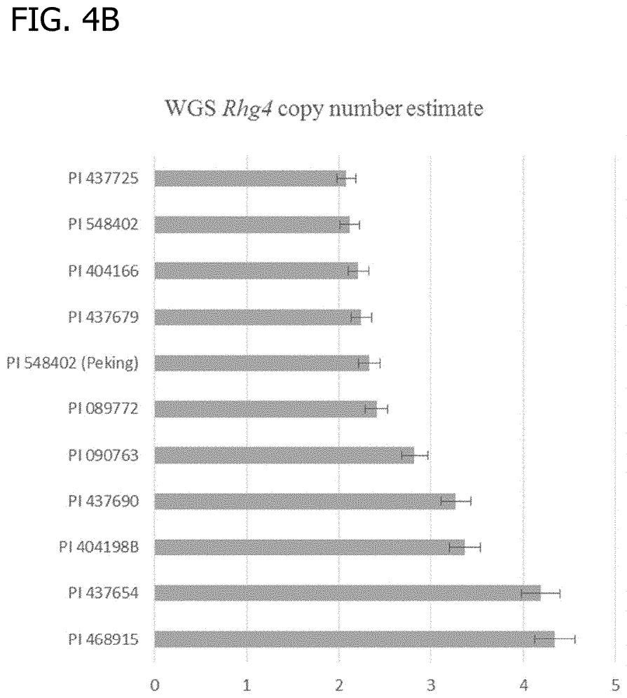

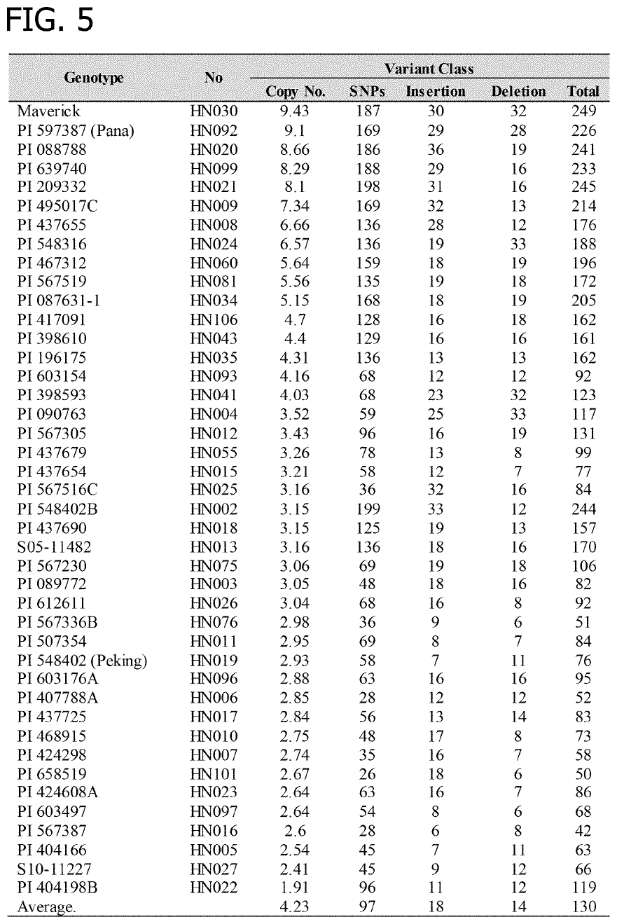

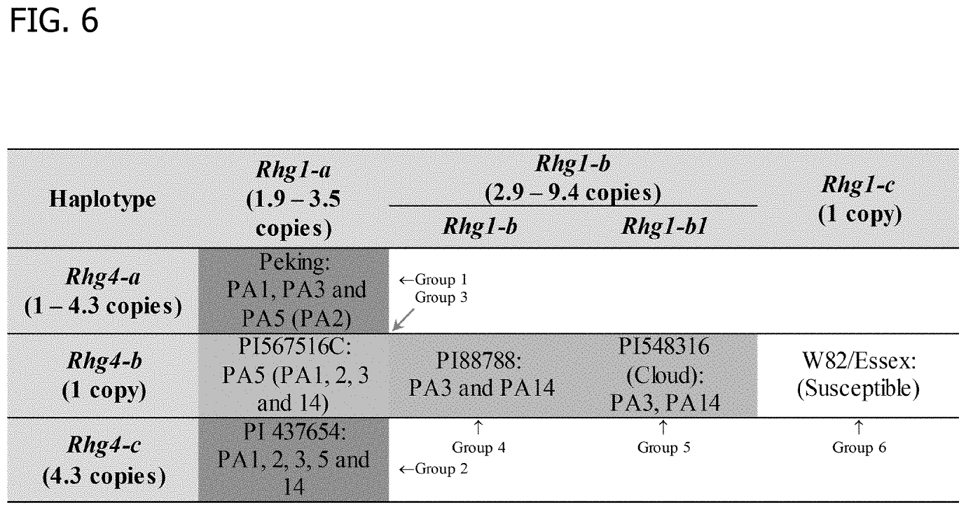

The genetic diversity at SCN resistance loci provided an opportunity to obtain an overview of the haplotype variation at both the Rhg1 and Rhg4 loci. As reported earlier, three genes (Glyma.18g022400, Glyma.18g022500 and Glyma.18g022600) at the Rhg1 locus together confer resistance to SCN in PI 88788 [Cook et al., 2012]. Despite a high number of sequence polymorphisms found within each Rhg1 repeat in SCN-resistant lines, the SNPs that cause an altered amino acid sequence (non-synonymous) were identified only in the Glyma.18g022500 (GmSNAP18) gene (FIG. 3). Three major haplotypes- named Rhg1-a, Rhg1-b and Rhg1-c- were identified for the GmSNAP18 gene based on ten amino acid sequences changes (Q203K, D208E, I238V, E285Q, D286Y, D286H, D287E, +287A (insertion of A residue after position 287), +287V (insertion of V residue after position 287), L288I) (FIG. 3). Additional beneficial amino acid changes not shown in FIG. 3 include A111D and +288T (insertion of T residue after position 288). The Rhg1-c corresponds to ‘Williams 82’-like Rhg1. The second haplotype was divided into Rhg1-b (similar to PI 88788-type lines) and Rhg1-b1 (similar to ‘Cloud’ type lines). Based on read depth across the known repeat and flanking regions, 45 lines were examined for CNV and showed an estimated Rhg1 copy number greater than one. The average number of copies across all tested lines was 3.6, with the highest at 9.4 for Maverick (FIG. 3 and FIG. 4A). Moreover, a wide range of DNA variation was observed at the Rhg1 locus, including SNPs, insertion, and deletion polymorphisms. Across the 25.1 kb interval, there was an average of 130 polymorphisms per accession compared with the soybean reference genome (FIG. 5). The patterns of amino acid variation at each Rhg1 genotype were highly correlated with the copy number and response to different SCN races. For example, the three major haplotype groups include high-copy Rhg1 (PI 88788-type, copy number from 2.9 to 9.4), low-copy Rhg1 (Peking'-type, copy number from 1.9 to 3.5) and single-copy Rhg1 (FIG. 6 and FIG. 3). The lines with high-copy number variation exclusively carry the PI 88788-type of SNP variants and the lines with low-copy number variation exclusively carry ‘Peking’-type of SNP variants. The lines with single copy Rhg1 do not carry any PI 88788- or Peking'-type of SNPs and are known to be susceptible to SCN.

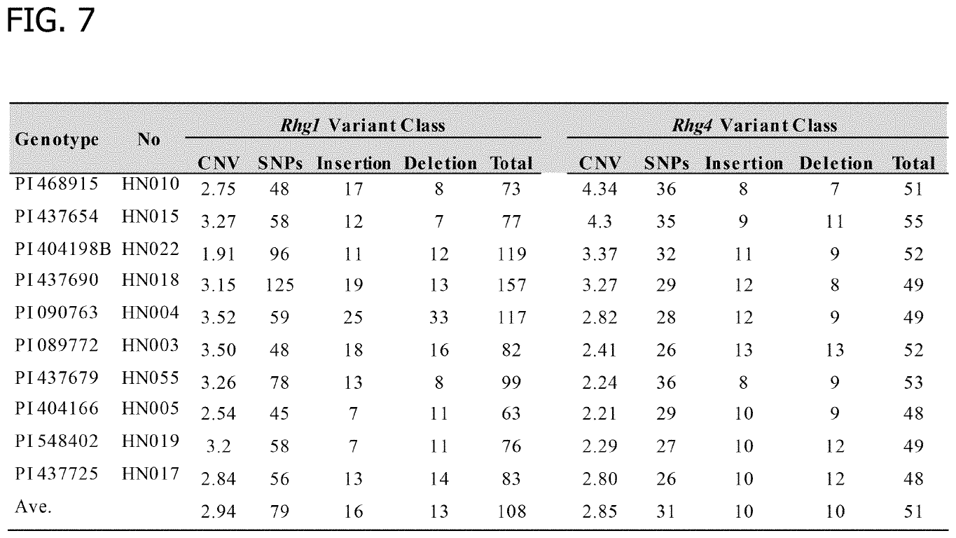

Similar to the Rhg1 locus, analysis of the sequence variation, CNV, and haplotypes at the Rhg4 locus encompassing three genes (Glyma. 08g108800, Glyma.08g108900 and Glyma.08g109000) was performed. The gene Glyma.08g108900, encoding Serine hydroxymethyltransferase (GmSHMT08), showed three nonsynonymous SNPs associated with the SCN reaction (FIG. 3). In the earlier soybean reference genome assembly W82.a1, GmSHMT08 (alias Glyma08g11490) was predicted to produce 503 amino acids, whereas in the most current assembly W82.a2 [Song et al., 2016] the primary transcript is 573 amino acids long. The first 70 amino acids in the assembly W82.al were missing, and this could be caused by an alternative splicing event or exon skipping. The CNV analysis showed the presence of multiple copies (1 to 4.3) of Rhg4, which were strongly associated with the non-synonymous SNPs leading to P<>R and N<>Y/H (FIG. 3). The highest number of Rhg4 copies was observed in PI 468915 and PI 437654. The average number of Rhg4 variant sites per soybean line was estimated to be 51 for multi-copy Rhg4 lines, and 26 for the single-copy Rhg4 lines in 21.3 kb interval compared to the reference genome (FIG. 7). Based on amino acid variants, the Rhg4 locus broadly divided into two haplotypes, the Rhg4-b (W82-like Rhg4) and Rhg4-a (‘Peking’-type Rhg4). Interestingly, PI 437654 carried additional non-synonymous SNPs leading to an I<>F amino acid change; this haplotype was named Rhg4-c (FIG. 3).

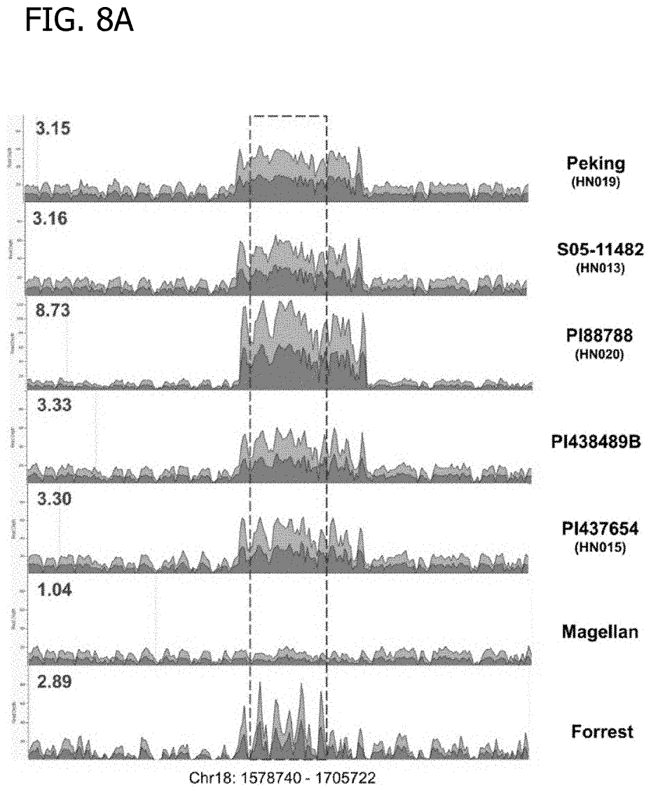

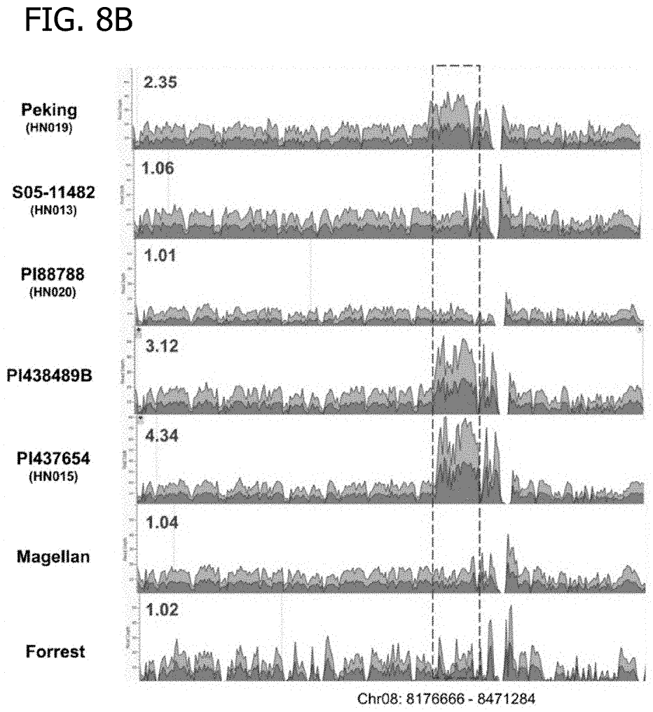

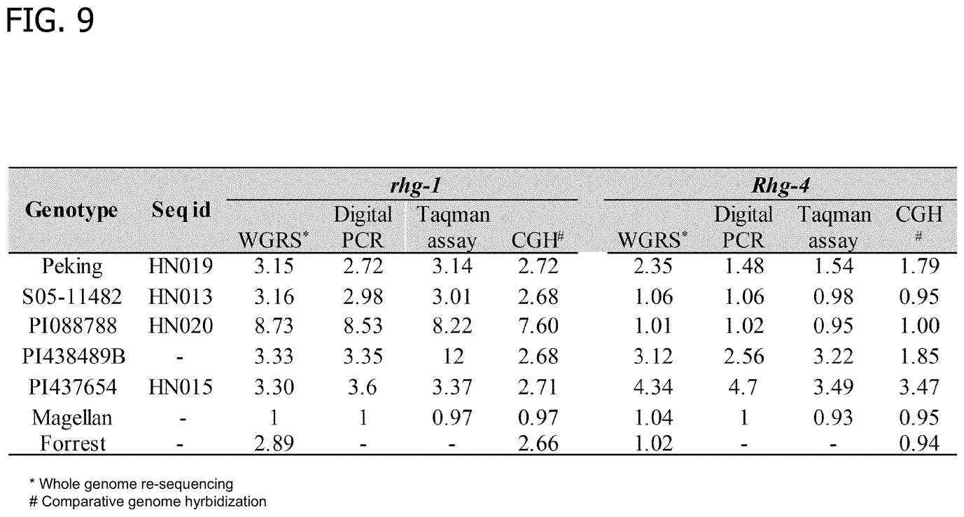

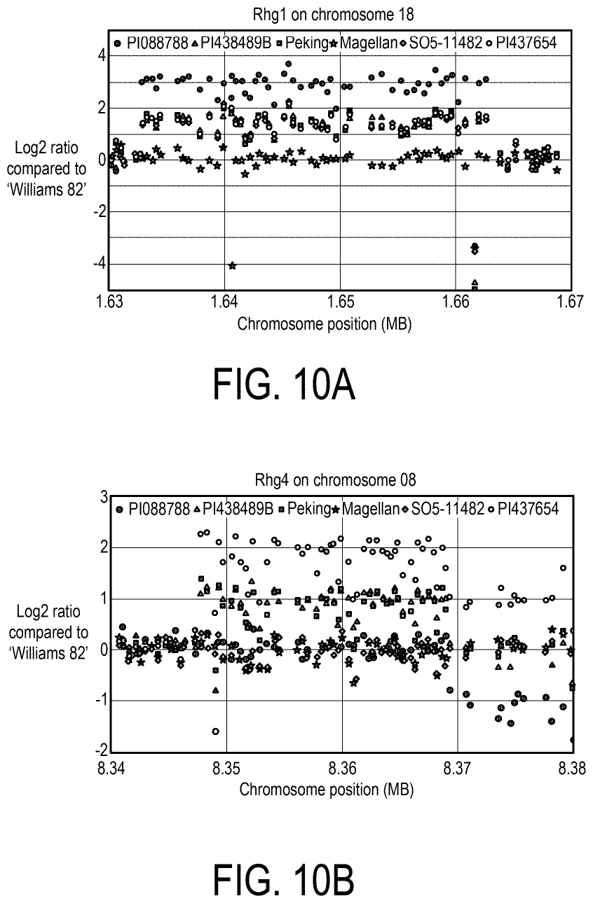

To further confirm the CNV estimated using WGRS data of both Rhg1 and Rhg4 loci (FIG. 8), additional experiments were performed, including Digital PCR, Taqman assays and microarray based comparative genomic hybridization (CGH) analysis (FIG. 9 and FIG. 10). Seven lines with known SCN resistance were selected for the verification of copy number at both Rhg1 and Rhg4 loci. The reported CNV data [Cook et al., 2012] for ‘Peking’, PI 88788, ‘Forrest’, PI 438489B, and PI 437654 were taken into consideration for comparison. Highly consistent results were observed across different platforms as well as earlier published studies (FIG. 9). Results obtained from the current study point to the first report showing the presence of CNV at the Rhg4 locus, directly impacting soybean cyst nematode resistance. Having established that both Rhg1 and Rhg4 have complex genomic and functional structures, additional experiments were planned to better resolve how the structural and functional properties interact in determining SCN resistance of soybean.

### Example 9

### SCN Epistatic Interaction Between Rhg 1 and Rhg4 Loci

Methods proceeded according to Examples 1-6, unless described otherwise.

Haplotype analysis revealed that only three non-synonymous SNPs at the GmSHMT08 gene showed a strong association with both CNV of Rhg4 loci and SCN resistance (FIG. 3). In this study, mutational analysis has been employed to study the impact of the three reported haplotypes representing the 106 sequenced soybean lines at important catalytic, substrate binding, structural stability, and subunit interaction sites within the GmSHMT08. The homology modeling was carried on ‘Forrest’ genotype, which carries three amino acid changes and also lacks the first 70 amino acids, suggesting that the first 70 amino acids do not affect the GmSHMT08 gene's function in resistance to SCN. The presence of 70 amino acids could be due to alternate splicing or exon skipping and these 70 amino acids might also have a role in organelle targeting, which warrants further study. The homology modeling analysis provided an interesting platform to study the differences between the resistant and susceptible haplotypes at GmSHMT08. Thus, the possible impact of each mutation on the interaction between all subunits of the putative GmSNAP18-GmSHMT08 complex was analyzed.

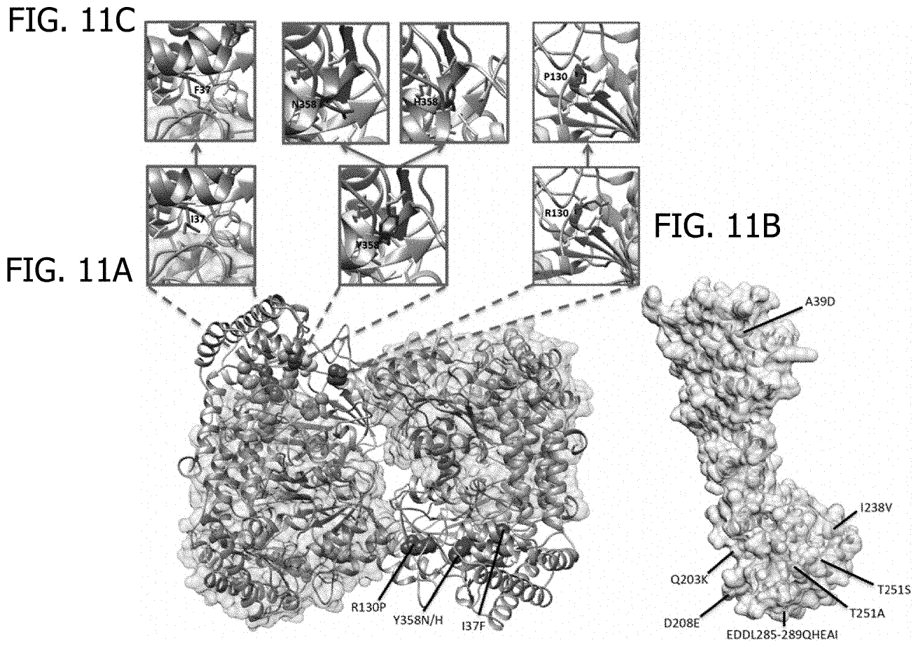

The protein homodimers play a critical role in catalysis and regulation through the formation of stable interfaces [Karthikraja et al.]. The homodimer-homodimer interface of the GmSHMT08 protein at P13OR (corresponding to P200R in FIG. 3) polymorphism is localized close to the pyridoxal phosphate (PLP) cofactor binding site and this site was specific to Rhg4-a and Rhg4-c alleles in SCN resistant lines. In addition, the amino acid change P130R (P200R in FIG. 3) leads change from a positively charged side chain arginine residue to an aliphatic uncharged proline residue, which is predicted to be involved in PLP cofactor binding. This mutation was shown to affect the tetramerization of the GmSHMT08 dimer and stability due to its suboptimal positioning that affects the binding events of the surrounding residues shown in five angstroms around the selected residue (FIG. 11). This spontaneous occurring mutation P130R affects 84.9% of the sequenced soybean lines. The third GmSHMT08 polymorphism (N389Y; N459Y in FIG. 3, which corresponds to N358Y in the Forrest line) represents 11.42% of the sequenced soybean lines and is not located at the dimerization site. However, this base resides within a pocket near the catalytic and substrate binding site of the GmSHMT08 protein, with a mutation directly altering the negatively charged hydrophobic tyrosine residue into a polar uncharged asparagine residue, which occurs in 86.66% of sequenced soybean lines (N389Y). This mutation was observed to present a major conflict with other residues (FIG. 11). However, a small fraction of the sequenced resistant soybean lines (1.98%) carried the Y389H (Y459H in FIG. 3, which corresponds to Y358H in the Forrest line) natural mutation; this polymorphism has no major effect with other residues since both tyrosine and histidine are an aromatic residue (FIG. 11). In the case of the I37F (I107F in FIG. 3), the amino acid change between two hydrophobic side chains; phenylalanine and isoleucine, presented no major conflicts with the other residues, as the observed positioning of residues surrounding the point mutation was conserved (in the 5 angstroms analyzed area) (FIG. 11). Only one soybean line (PI 437654) carried this polymorphism among the 106 sequenced lines.

### Example 10

### Identification of Tandem Repeats at the Rhg4 Locus

Methods proceeded according to Examples 1-6, unless described otherwise.

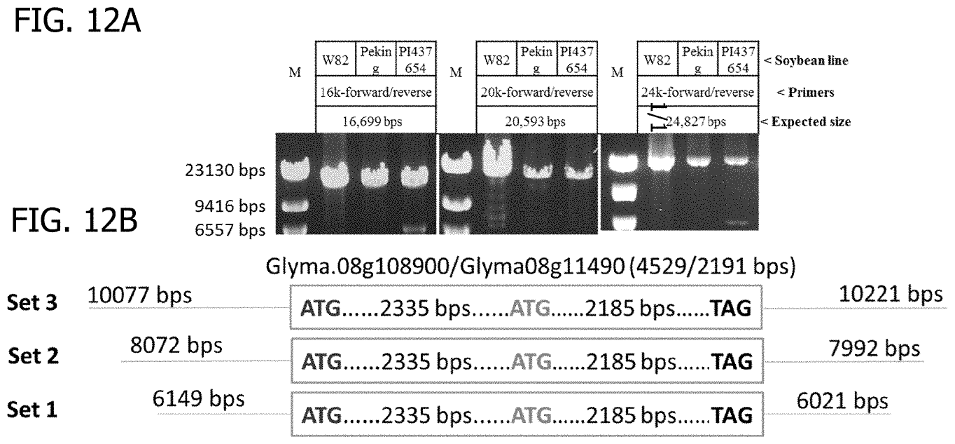

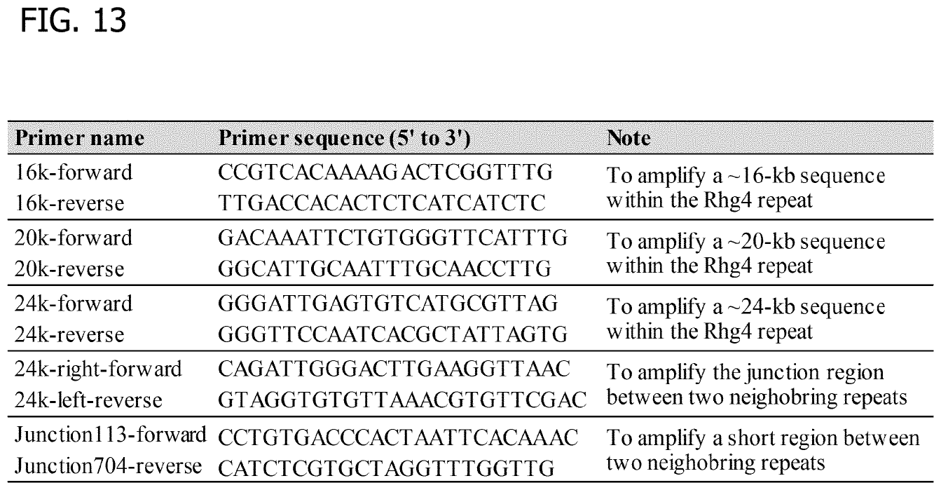

Based on the WGRS information, the genomic region surrounding the cloned Rhg4 gene GmSHMT08 [Liu et al., 2012] appeared to be duplicated in at least 11 of the 106 sequenced genomes (FIG. 3). This finding was confirmed in ‘Peking’, PI 437654 and PI 438489B using a combination of CGH, DPCR, and Taqman assays (FIG. 9). The duplicated region was estimated to be approximately 30-kb (FIG. 12). To confirm whether the duplications are indeed present in these lines and to reveal their sizes and locations, three sets of primers were first designed based on the reference genome of ‘Williams 82’ to see whether experiments could amplify 16.7-kb, 20.6-kb, and 24.8-kb regions flanking the cloned Rhg4 gene. Results obtained hypothesize that if two primers are located inside a complete duplicated region, a PCR product of the expected size defined by the primers should be generated. Indeed, after the PCR amplification, a PCR band of the expected size was detected in ‘Williams 82’, ‘Peking’ and PI 437654 for all three-primer sets, respectively (FIG. 13). These results suggest that these primers as well as the regions defined by them are located inside a duplicated region (if such a duplication exists in a given genotype), and that the duplicated region or repeat should be longer than the 24.8-kb region.

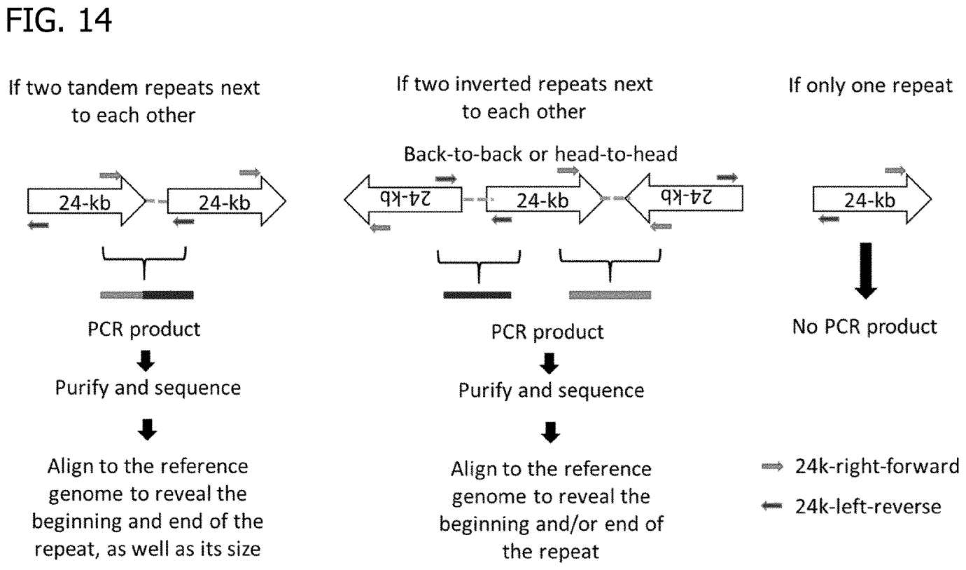

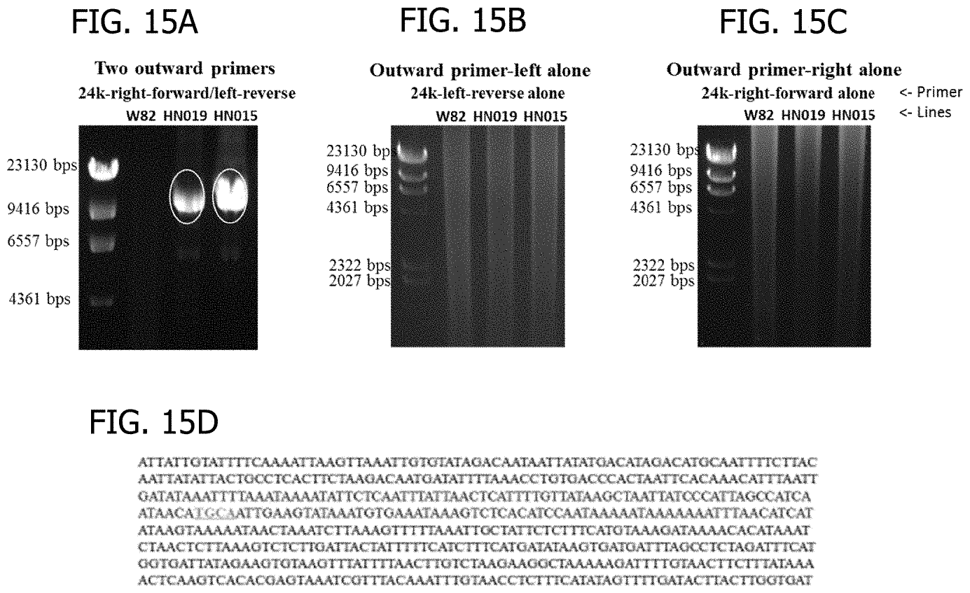

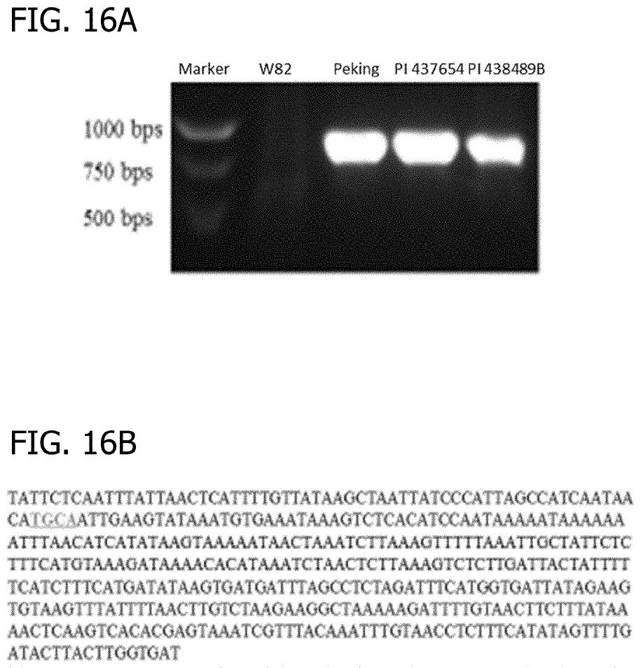

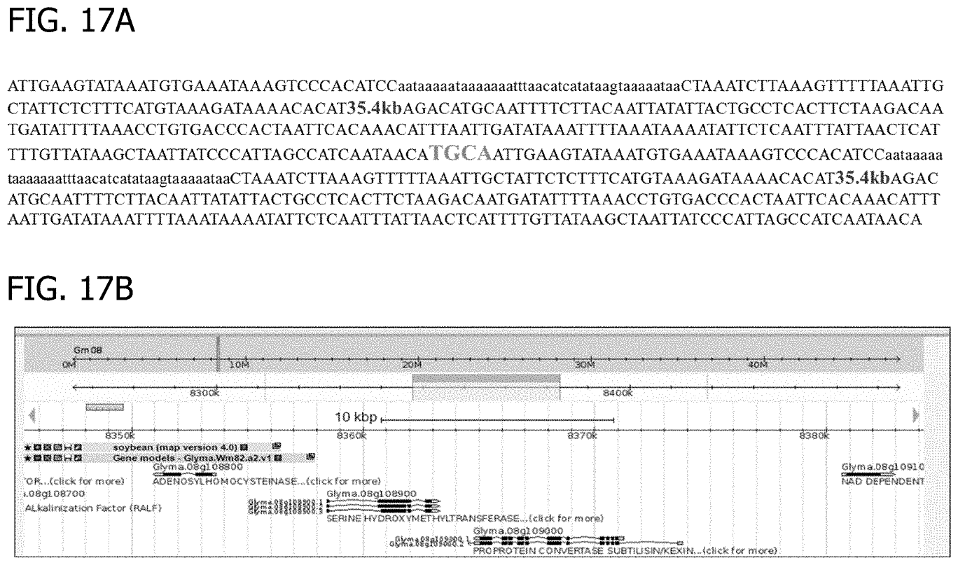

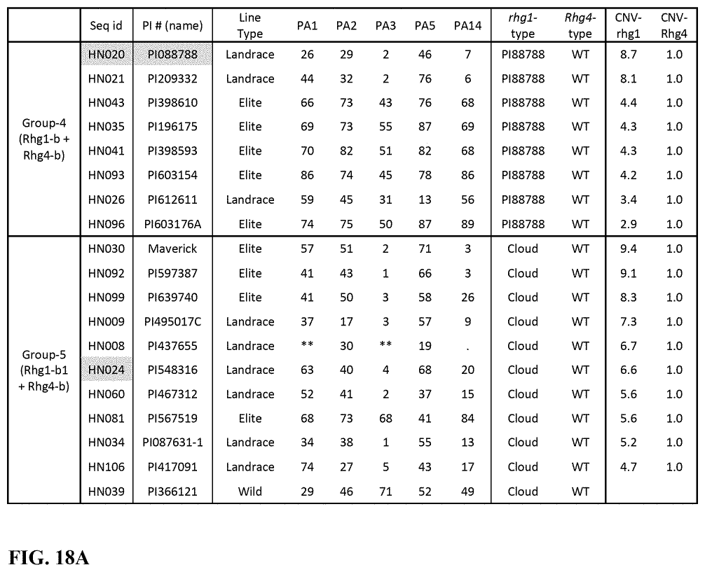

Since this 24.8-kb length is rather close to the estimated 30-kb duplicated region, it was speculated that the ends of this 24.8-kb region were likely close to the junction between two neighboring repeats. If this is the case, it may be possible to amplify by PCR this junction region in the lines with duplications using two outward end primers of the 24.8-kb region as depicted graphically in FIG. 12 and FIG. 14. However, these primers should fail to amplify in ‘Williams 82’, which does not have any duplication at the Rhg4 locus. Indeed, a PCR band of approximately 11-kb was generated in both ‘Peking’ and PI 437654, but not in Williams 82, when both primers were included in the reactions (FIG. 15). No PCR bands were generated in any lines when a single outward primer was used in the reactions, which were intended to amplify the junctions between two neighboring inverted (either back-to-back or head-to-head) repeats (FIG. 15). After sequencing the purified PCR products from both lines, two sequences from different locations of the reference genome were found linked with each other, separated by the following four base pairs: TGCA (FIG. 15). The joining of two sequences from different regions in these lines indicates that duplications or sequence arrangements are present. To confirm that the obtained junction sequence was not due to PCR artifacts, two primers were designed to flank an 819-bp junction region and were used in PCR reactions on genomic DNA from different soybean lines. After PCR amplification, a PCR band of approximately 800 bp was detected in ‘Peking’, PI 437654, and PI 438489B, but not in ‘Williams 82’. Most importantly, the sequences obtained from these PCR products matched the initially identified junction sequence (FIG. 16). Therefore, experiments support that repeats are present in these lines and the sequence upstream the TGCA should correspond to the end of one repeat and the sequence downstream the TGCA should be the beginning of the neighboring tandem repeat (in the same orientation as 24.8-kb region). By aligning the beginning and end sequences with the reference genome, it was found that the repeat at the Rhg4 locus in ‘Peking’, PI 437654, and PI 438489B was 35,705 bp (FIG. 17). Interestingly, according to the reference genome, this repeat contains the following four genes, Glyma.08g108800 (Adenosylhomocysteinase), Glyma. 08g108900 (the cloned Rhg4, encoding a serine hydroxymethyltransferase, SHMT), Glyma. 08g109000 (encoding a proprotein convertase subtilisin/kexin), and Glyma. 08g109100 (encoding a NAD dependent epimerase/dehydratase) (FIG. 17). It should be noted that the PCR analysis provides the structural map for at least one junction in the tandem repeat arrangement, but does not confirm that all copies from all of the genotypes have the same structure.

### Example 11

### Rhg4 Copy Number and Broad-Based Resistance to SCN

Methods proceeded according to Examples 1-6, unless described otherwise.

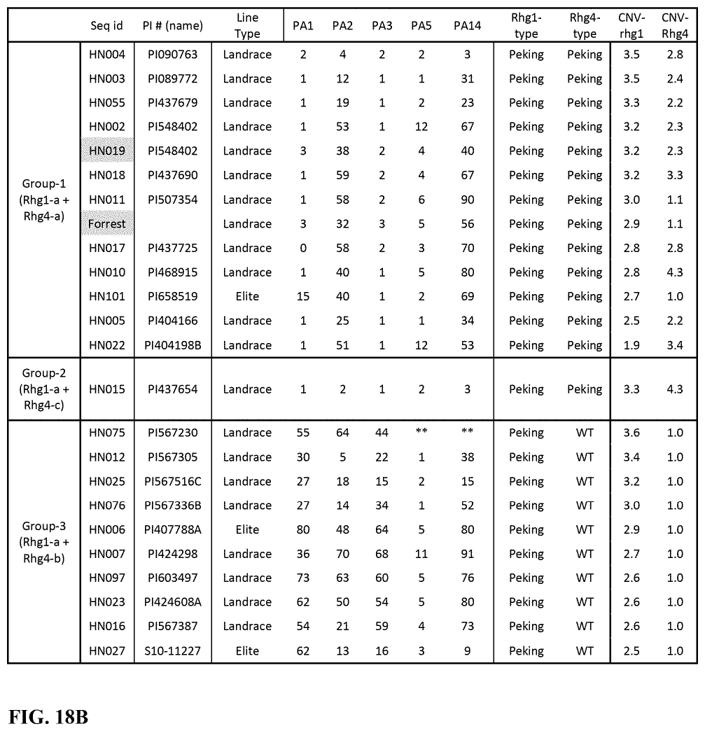

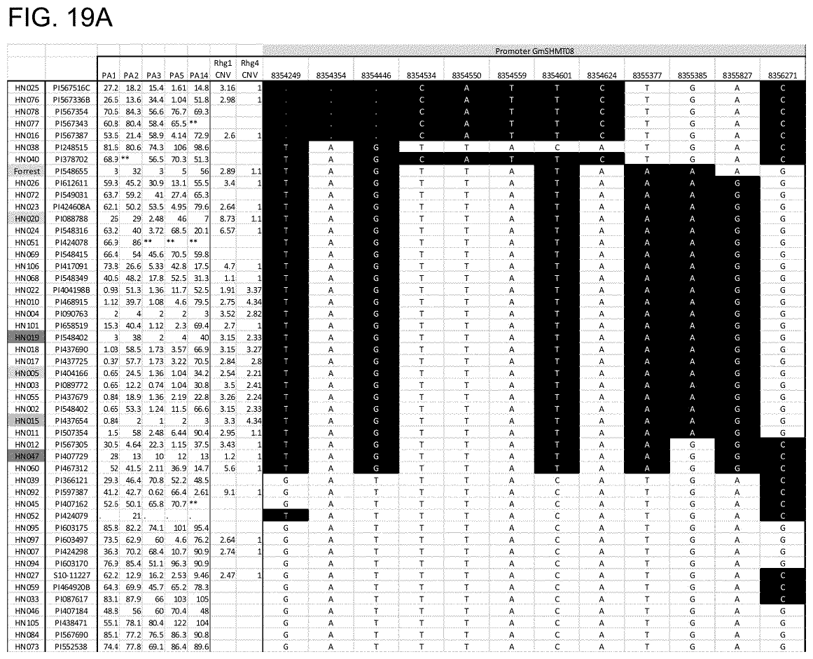

The presence of CNV for the Rhg1 locus is common (or frequent) when compared to the Rhg4 locus (FIG. 3 and FIG. 18) and the PI 88788 source carrying high copies of Rhg1 is used in over 95% of existing SCN resistant varieties marketed in the US. However, the PI 88788-type resistance has been broken down due to adaptation in SCN populations. Several lines carrying the haplotypes Rhgl-b or Rhgl-bl, and having greater than 5.6 copies of the GmSNAP18 showed SCN resistance to race 3 and 14. The remaining lines with Rhg1-b or Rhg1-b1 but less than 5.6 Rhg1 copies were susceptible to three to four SCN races, except PI 417091 (FIG. 3). Thus, a copy number of 5.6 of Rhg1 can be hypothesized to be the threshold for resistance to both races 3 and 14. These lines do not carry CNV or nonsynonymous mutation in the GmSHMT08 gene. However, lines carrying ‘Peking’-type Rhg1 (Rhg 1-a haplotype) with relatively lower copies (1.9 to 3.5) showed resistance to multiple SCN races. This is because these lines also carry CNV and/or retained nonsynonymous mutations in GmSHMT08 (i.e. Rhg4-c and Rhg4-a) (FIG. 3 and FIG. 6). For example, PI 567516C carry not only the Rhg 1-a allele, but also carries the wild-type allele at Rhg4 (Rhg4-b), and hence showed moderate resistance to multiple races. However, a line (e.g. PI 437654) carrying multiple copies of Rhg-4 in addition to Rhg1-a oftentimes showed resistance to all five races. From these observations, it follows that in addition to Peking'-type GmSNAP18 with 2 to 4 copies, the CNV and nonsynonymous SNPs in the GmSHMT08 gene play a paramount role to gain resistance to multiple races.

Based on epistatic interactions of the GmSNAP18 and GmSHMT08, the 106 soybean lines were grouped into six categories that showed strong associations between genotypic variation (CNV and non-synonymous changes) and nematode susceptibility/resistance phenotypes (FIG. 6 and FIG. 18). The lines of group-1 and -2 (Rhg1-a+Rhg4-a and Rhg1-a+Rhg4-c, respectively) carry only Peking'-type of Rhg1 and Rhg4 and were highly resistant to race 1, 2, 3, 5, and resistant or moderate resistant to race 14. Lines belonging to group-3 (Rhg1-a+Rhg4-b) carry only Peking'-type Rhg1 and conferred resistance to race 5. The group 4 and 5 (Rhg1-b +Rhg4-b and Rhgl-b1 +Rhg4-b, respectively) lines carry only PI 88788/'Cloud'-type of the Rhg1 and showed greater resistance to races 3 and 14. A comparison of PI 88788 and ‘Cloud’ type Rhg1 indicated that the lines with the ‘Cloud’-type of Rhg1 performed better resistance. The lines belonging to the group-6 (Rhgl-c+Rhg4-b) carry ‘Williams 82’-type loci and hence were highly susceptible to all five SCN races (FIG. 18). Surprisingly, PI 407729 (a group 6 line) does not carry the above-mentioned resistant loci (non-synonymous SNP and CNV), but exhibited moderate to high resistance to all five races. These observations suggest that this line may contain novel resistance loci that confer SCN resistance independent of Rhg1 and Rhg4. To infer the resistance mechanism in PI 407729, GmSHMT08 and GmSNAP18 promoter haplotypes were analyzed as discussed in the next sections.

### Example 12

### Variation in GmSHMT08 and GmSNAP18 Promoters in Combination with CNV Confers Additional Level of Resistance to SCN

Methods proceeded according to Examples 1-6, unless described otherwise.

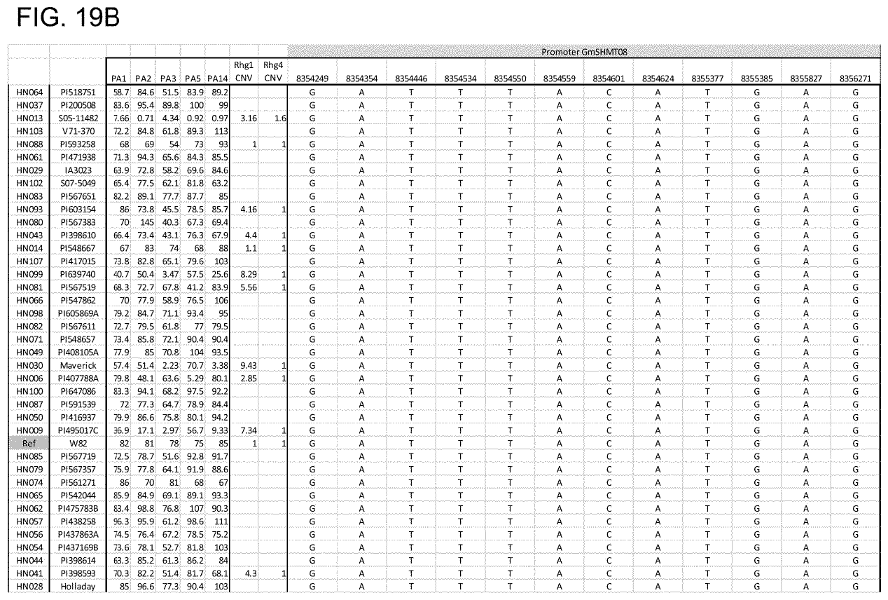

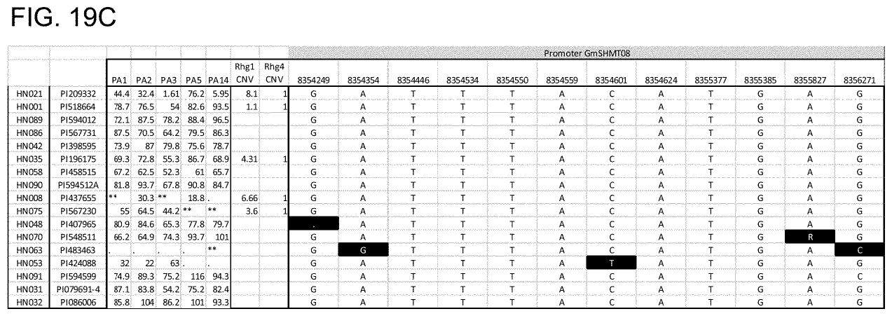

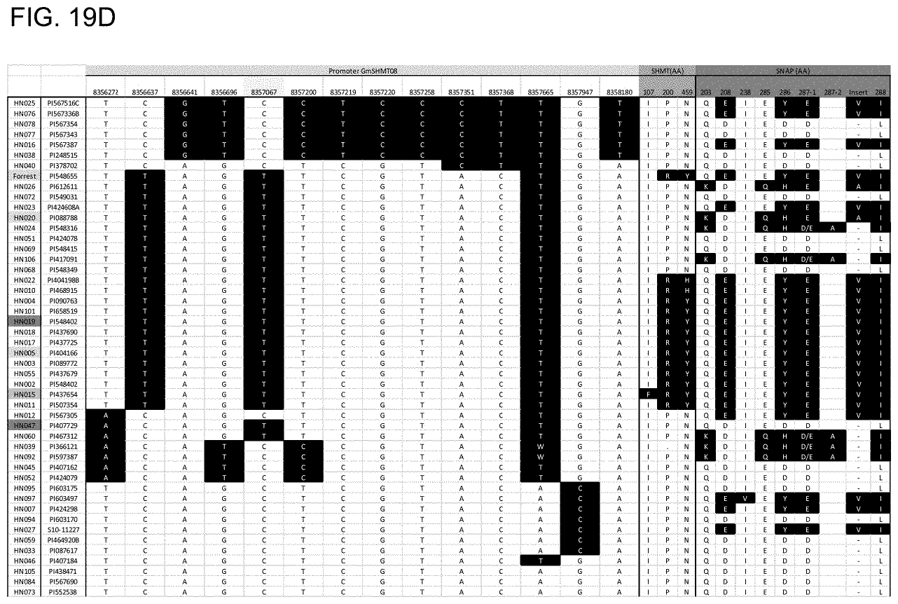

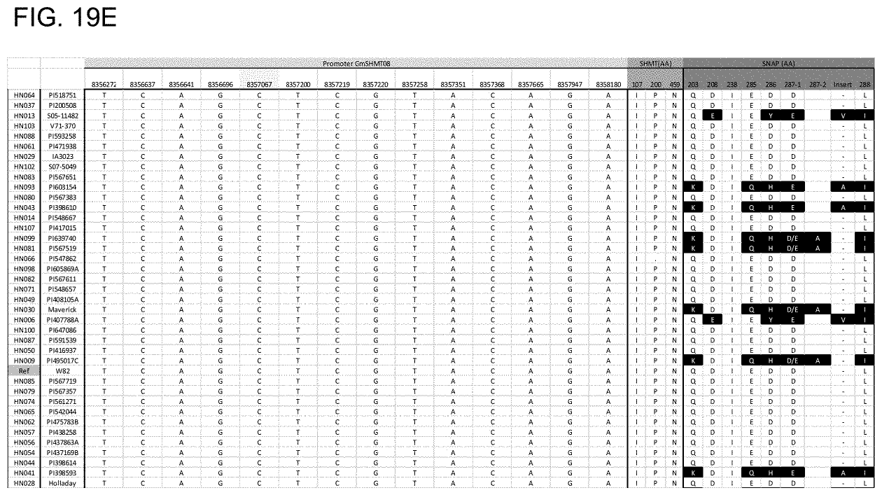

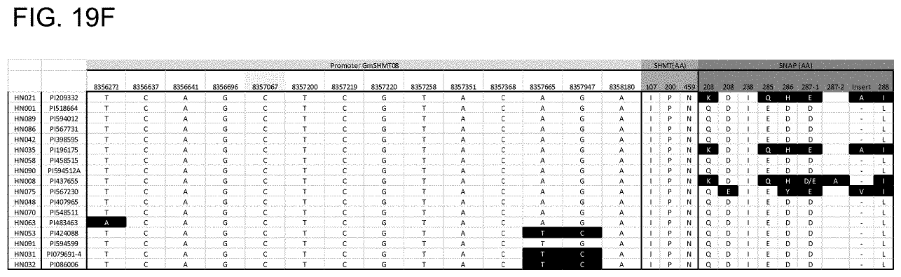

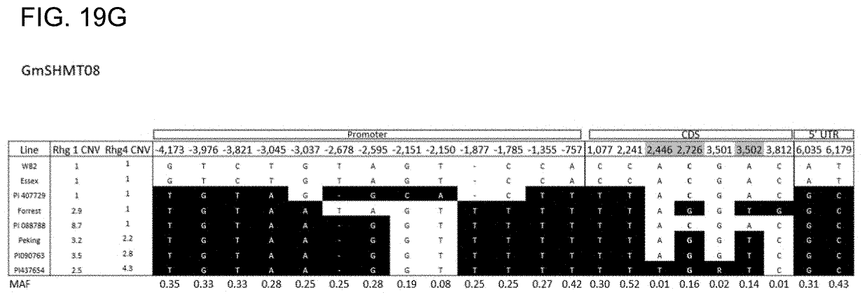

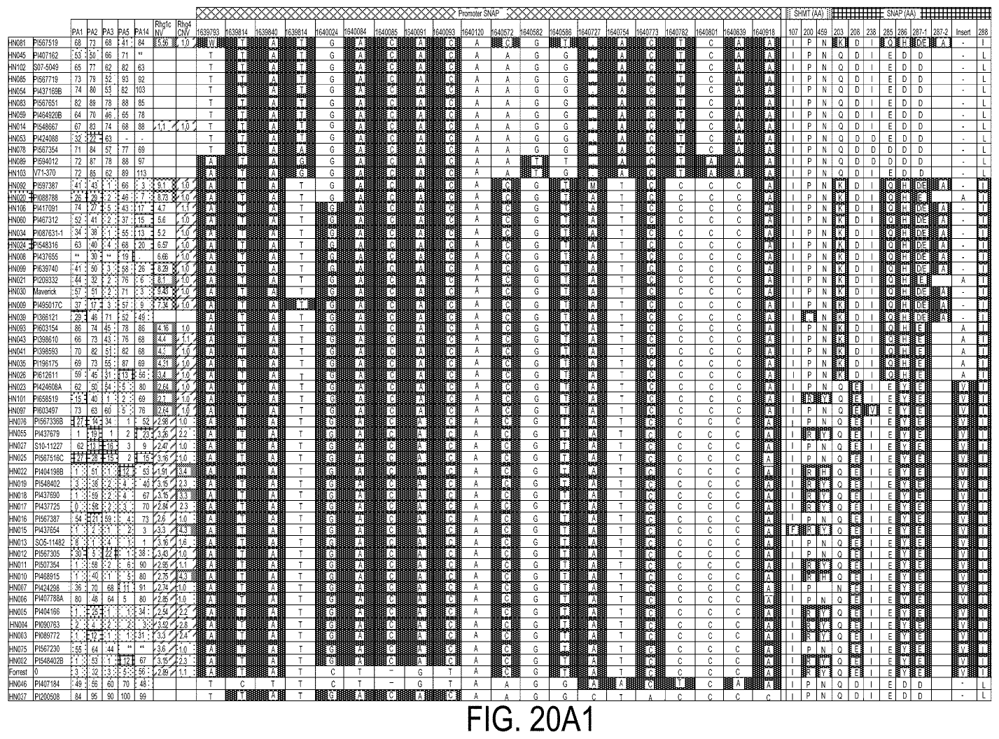

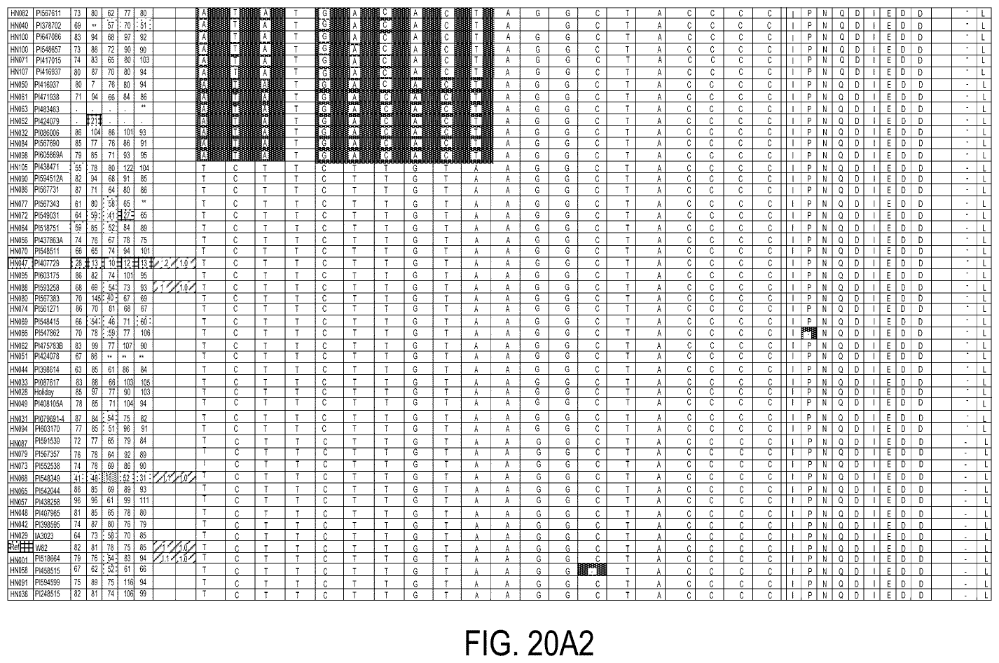

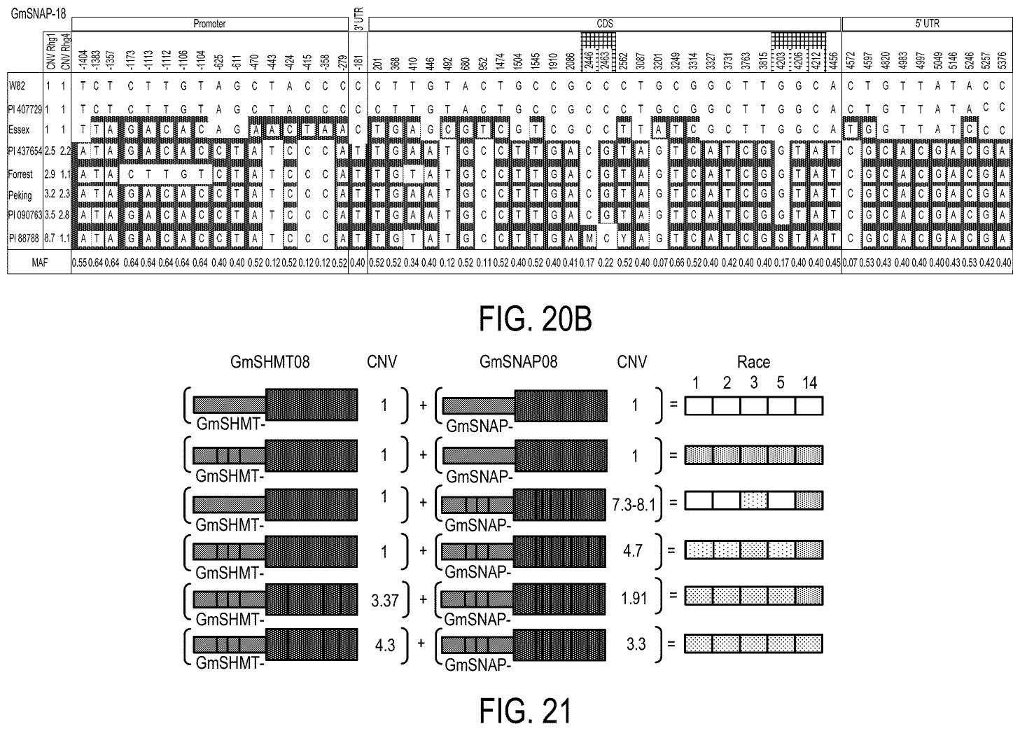

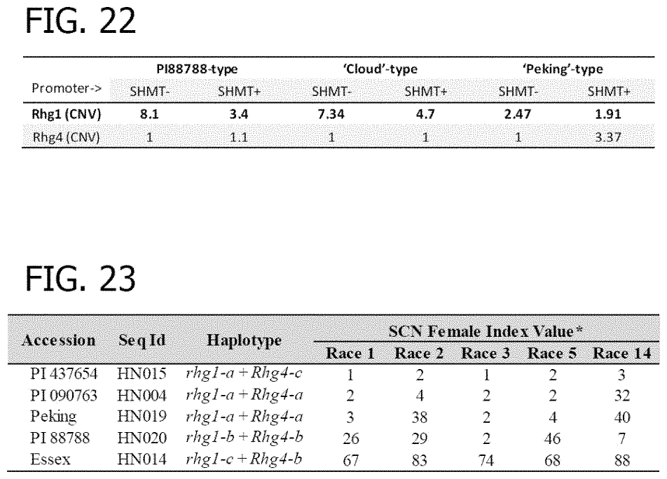

These Examples have shown that resistant alleles contain either nine or three natural point mutations in the GmSNAP18 and GmSHMT08 proteins, respectively, when compared to the susceptible alleles. Out of the 106 lines examined, 14 lines carry resistant alleles at both the Rhgl-a and the Rhg4-a/Rhg4-c haplotypes, corresponding to the Peking'-type of resistance. However, the other 30 SCN resistant lines, corresponding to both ‘Cloud’- and PI 88788-type of resistance, carry the resistant Rhg1-a (11 lines), Rhg1-b (8 lines), and Rhgl-b1 (11 lines) haplotype, but all contain the Rhg4-b susceptible allele. Interestingly, PI 407729 carries both susceptible alleles at the Rhg1-c and the Rhg4-b loci, but exhibited resistance to all five races. In order to gain more insight into SCN resistance in this line, a haplotype analysis clustering of all the 106 lines at the promoter level of both genes was performed (FIG. 19 and FIG. 20). It is well documented that SNPs in the promoter region, including the 5′ UTR, can abolish gene function, expression level, and localization [Patil et al., 2015]. The analysis suggested an additional layer for the resistance mechanism. In fact, the haplotype of the GmSHMT08 promoter region (˜3.8 Kb) showed that most of the resistant lines carry a unique haplotype, which was different from that of the SCN susceptible lines. Moreover, the analysis substantiated that PI 407729 carries several SNPs and Indels in the promoter region that are different from the susceptible lines ‘Williams 82’ and ‘Essex’, but similar to the promoters of the resistant lines (GmSHMT08⁺) ‘Forrest’, ‘Peking’, PI 88788, and PI 437654. This observation suggests that the SNPs/indels identified in the GmSHMT08⁺ promoter may be responsible for SCN resistance in PI 407729 (FIG. 19 and FIG. 21). Notably, copy numbers of 3.4 and 4.7 were enough to confer broad-based resistance to SCN when the GmSHMT08⁺ promoter is present. However, if a given soybean line lacks the GmSHMT08⁺ promoter, then at least 8.1 and 7.3 copies of the GmSNAP18 (Rhg1) are required to confer resistance in PI 88788- and ‘Cloud’-type-Rhg1, respectively (FIG. 22). Similarly, in the case of Peking'-type lines, 1.91 copies of Rhg1 are enough to confer SCN resistance when the GmSHMT08⁺ promoter is present. However, when the promoter variation (GmSHMT08⁻) is present, the Rhg1 copy number should be at least 2.47 in order to confer resistance to SCN (FIG. 19, FIG. 20, FIG. 21, and FIG. 22).

Similarly, the haplotype analysis of GmSNAP18 promoter (˜1.5 Kb) showed that the majority of the resistant lines carry a specific promoter haplotype (FIG. 20 and FIG. 21). In addition, lines that lack this promoter haplotype were found to be susceptible to SCN. Interestingly, four lines PI 196175, PI 398593, PI 398610 and PI 603154 carry both the resistant loci (non-synonymous SNP and CNV at the Rhg1 locus) and promoter haplotype but were found to be susceptible to SCN. This can be explained by presence of the susceptible GmSHMT08⁻ promoter. Overall, these results suggest that variants (SNP/indel) within the promoter region coupled with CNV provides an additional layer of resistance, and the susceptible lines may be converted into resistant by replacing the susceptible promoter with the GmSHMT08⁺ version (FIG. 21).

### Example 13

### Expression Analysis and Rhg4/Rhg1 Copy Number Variants

Methods proceeded according to Examples 1-6, unless described otherwise.

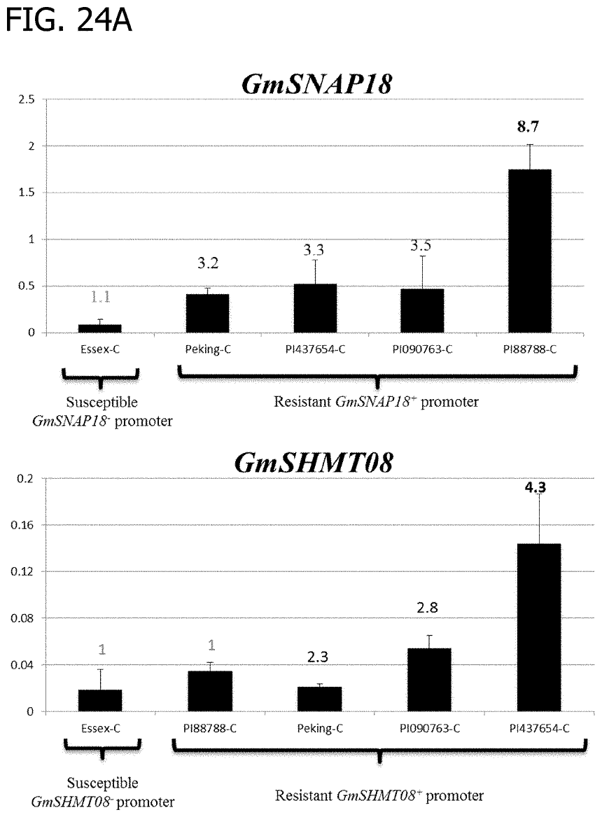

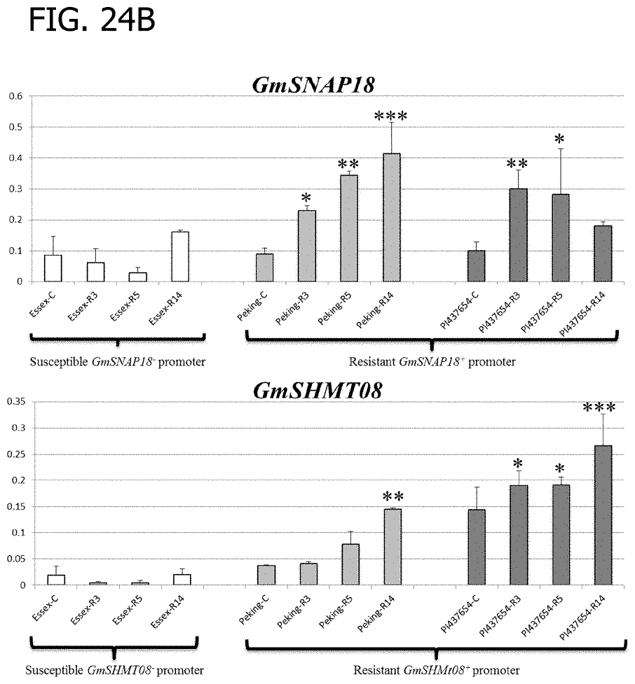

To gain more insight into the impact of the identified CNV on both the GmSNAP18 and GmSHMT08 transcripts, qRT-PCR analysis was carried out in a number of lines representing different subgroups. Based on the haplotype combinations and CNV, five indicator lines including ‘Essex’, ‘Peking’, PI 437654, PI 090763, and PI 88788 were selected, and screened in the presence and absence of the nematode infection (FIG. 23). In the absence of SCN infection, expression analysis shows that the GmSNAP18 root transcripts in five indicator lines correlates perfectly with their Rhg1 CNV (FIG. 24A). In fact, GmSNAP18 transcripts in PI 88788, which has the highest copy number (8.7) of Rhgl, were 2.70, 2.34, 3.24, and 20.75 times more abundant when compared to PI 090763 (copy number=3.5), PI437654 (copy number=3.3), ‘Peking’ (copy number=3.2), and ‘Essex’ (copy number=1.1), respectively. Overall, GmSNAP18 transcripts were up to 10-fold more abundant than the GmSHMT08 transcripts. Notably, the tested lines also carry SNP in the GmSHMT08⁺ promoter (FIG. 24A). In the case of GmSHMT08, PI 437654 has the highest Rhg4 copy number (4.3) and exhibited 1.8- and 6-fold more abundant transcripts when compared to PI 090763 (copy number=2.8) and ‘Peking’ (copy number=2.3), respectively. In addition, PI 437654 transcripts were 13-fold more abundant than ‘Essex’ (copy number=1) carrying the susceptible GmSHMT08⁻ promoter. In summary, the obtained results show that both the promoter variation and copy number are associated with the differences in Rhg4 gene expression.

Recently, it has been shown that GmSNAP18 transcripts were induced in ‘Forrest’ (carrying the Rhg1-a and Rhg4-a haplotypes) and PI 88788 (carrying the Rhg1-b and Rhg4-b haplotypes) in response to SCN infection, whereas the susceptible line ‘Essex’ (carrying the Rhg1-c and Rhg4-b haplotypes) showed very low mRNA levels of GmSNAP18 [Liu et al., 2017]. In Forrest, GmSNAP18 transcripts showed about 2-fold upregulation in SCN-infected roots compared to non-infected roots at 3 and 5 days post SCN infection (dpi). Similarly, in PI 88788 GmSNAP18 transcripts showed 2-fold upregulation in SCN infected root compared to non-infected control at 5 dpi. GmSHMT08 transcripts were also found to be induced in both ‘Forrest’ and PI 88788 soybean lines [Kandoth et al.]. Similarly, the expression of ‘Essex’, ‘Peking’, and PI 436754 in response to infection by three SCN races (PA3, PA5, and PA14) at 2 dpi was investigated. The analysis demonstrated that GmSNAP18 transcripts (underlying Rhgl-a haplotype) were induced in the presence of the three nematode races in both ‘Peking’ and PI 436754 (FIG. 24B). In summary, all the resistant lines tested and carrying the Rhg1-a, Rhg1-b, Rhg4-a, Rhg4-b, and Rhg4-c haplotypes exhibited abundant transcripts in the absence of SCN infection, a finding that correlates with the CNV in these lines. In addition, their transcript levels were further induced in the presence of the three SCN races tested. However, susceptible lines like ‘Essex’ with reduced copy number (Rhgl-c=1.1 and Rhg4-b=1) exhibited the lowest expression level and absence of any induction of the Rhg1-c nor Rhg4-b transcripts.

### Example 14

### Haplotype Analysis

Methods proceeded according to Examples 1-6, unless described otherwise.

Soybean germplasm provides a wide range of SCN resistance that is controlled by natural variants (SNP and CNV) at two major loci, Rhg1 and Rhg4. In these Examples, high-quality deep sequencing information (˜15× genome coverage) for the Rhg1 and Rhg4 loci were utilized and haplotypes associated with SCN resistance to five races were identified. Haplotype analysis also identified SNPs associated with CNV. The CNV of the Rhg1 alleles, which carries 2 to 10 copies across different soybean varieties, is a well-known phenomenon [Lee et al.; Cook et al., 2014]. It is not surprising that nearly identical results for CNV of the Rhg1 locus were obtained, which is also related to the SCN-resistant efficacy, as previously reported. It was interesting, however, that increased copy number of the Rhg4 gene was observed in 11 soybean lines, ranging from 1.2 to 4.3 copies. The copy number increases were confirmed using different molecular platforms, including Digital-PCR, Taqman assay and CGH. Furthermore, a tandem repeat structure at the Rhg4 locus was also confirmed. A sequence of 35.7-kb was found duplicated at the Rhg4 locus in ‘Peking’, PI 437654 and PI 438489B. The duplicated region contains four genes, including the cloned Rhg4 gene, which encodes a serine hydroxymethyltransferase (SHMT). This new discovery provides a new insight for the SCN resistance mechanism at the Rhg4 locus.

During the last decade, many studies examined segmental duplication and genome re-sequencing applications, with a special focus on the identification of CNVs [Zarrei et al.; Sharp et al.; de Koning et al.]. In fact, deletions and duplications are considered to be major contributions to the genome variability, playing important roles in generating variation among many traits, including disease phenotypes. Many studies explored the human genomes for genetic disorders and identified a range of variants [Inoue & Lupski; Perry et al., 2007; Myers; Albertini et al.; Macdonald et al.]. However, CNV is an important type of structural variation because of its varied evolutionary impacts, stimulating genomic rearrangements, and gene dosage effects [Olsen & Wendel; Moore & Purugganan; Flagel & Wendel]. Different types of CNV have been observed in diverse organisms, including humans and chimpanzees [Perry et al., 2008], rats [Aitman et al.], Arabidopsis [DeBolt], extremophile crucifer [Dassanayake et al.] and Plasmodium falciparum [Heinberg et al.]. In soybean, it has been previously reported that copy number of three genes together, at the Rhgl-b locus, encoding a Soluble NSF Attachment Protein (a-SNAP), an Amino Acid Transporter (AAT), and a Wound-Inducible domain (WI12), mediates nematode resistance in soybean PI 88788 type of resistance [Cook et al., 2012; Bayless et al., 2018]. These Examples provide strong evidence that CNV of GmSHMT08 at the Rhg4 locus also plays a significant role in SCN resistance. Interestingly, mutations in human SHMT have been linked to a wide range of diseases [Maddocks et al.; Skibola et al.; Lim et al.]. Moreover, an shmt knockout mutant was shown to induce apoptosis in lung cancer cells by causing uracil misincorporation [Paone et al.]. Therefore, the findings on SHMT allelic variation in these Examples may have implications beyond the field of plant pathology, as similar variants may be important within the field of pharmacogenomics due to SHMT's involvement in human cancer.

These Examples demonstrated that the resistant allele contains three critical spontaneously occurring natural point mutations resulting in four amino acid changes; I37F (0.94%), P13OR (15.1%), N358Y (11.32), and Y358H (1.88%) at the GmSHMT08 protein when compared to the susceptible alleles. Homology modeling suggests that these point mutations may impair the key regulatory property of the encoded GmSHMT08 enzyme, including subunit associations (Dimerization and tetramerization), PLP cofactor and substrate binding, and catalytic site. The altered enzyme may further influence the folate homeostasis in soybean root cells, and ultimately restrict the growth of cyst nematodes in susceptible soybean lines, as has been suggested previously [Liu et al., 2012]. The current study demonstrated that the resistant Rhg4 allele was detected in 13.2% of the sequenced soybean lines representing the USDA Soybean Germplasm Collection, including ‘Peking’. Additionally, it has been reported that overexpression of Rhg4-‘Peking’ in roots of SCN-susceptible cultivar ‘Williams 82’ greatly reduced nematode parasitism [Matthews et al.].

### Example 15

### Limited Haplotypes and SCN Resistance in the U.S. Germplasm

Methods were according to Examples 1-6, unless described otherwise.

Since the discovery of SCN resistance QTL, most of the varieties in the U.S. trace back to ‘Peking’- and/or PI 88788-type of resistance. Due to the effectiveness of the high copy Rhg1 from PI 88788 source, it was frequently utilized (over 95%) by breeders to develop elite cultivars. However, limited variation, especially at the Rhg4 locus was captured in the recent breeding programs. The effectiveness of PI 88788-type resistance is breaking down due to continuous cropping of soybean varieties derived from PI 88788. Another reason could be that the Rhg1-type of resistance was sufficient at the time of development. However, due to virulence and adaptation of SCN populations, the high copy Rhg1 is not sufficient to confer broad-based resistance unless a new epistatically interacting (additive) resistant haplotype is substituted. The lack of genetic diversity and/or the right combination of resistant haplotypes has led to a widespread shift towards virulence in SCN populations. Analysis from these Examples showed that susceptibility phenotypes associated with low copies of Rhg1 could be overcome by incorporating Rhg4 alleles.

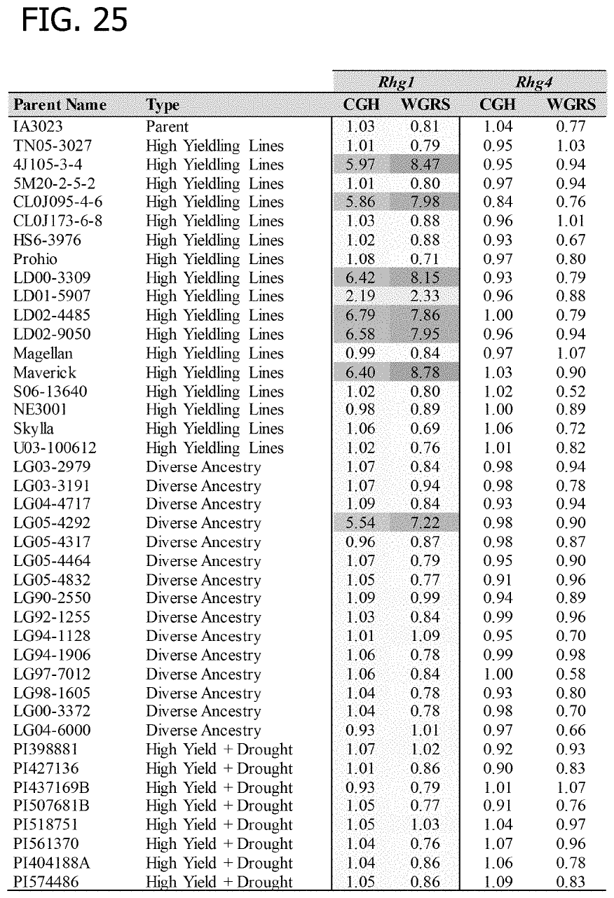

The 106 WGRS set contains 57 elites, 44 landraces, and 7 wild soybean lines [Valliyodan et al.]. None of the elite lines carry multiple copies at the Rhg4 locus and most of the lines (49/57) were highly susceptible to two or more SCN races (FIG. 18). To further confirm this result the whole genome sequence and CGH data from soybean NAM (Nested Association Mapping) population [Song et al., 2017] was utilized and CNV was estimated [Anderson et al.] (FIG. 25). The soybean NAM populations consist of 17 high-yielding lines from eight states from the U.S., 15 lines with diverse ancestry, 8 lines are exotic PIs, in addition to the cv. ‘IA3023’, which was used as common parent for crossing with all 40 lines. Interestingly, 8 out of 41 parents carry more than two copies of the Rhg1 locus with maximum of 6.79 copies in LD02-4485. However, in case of the Rhg4 locus, no CNV was observed. This observation suggests that a limited number of resistant haplotypes was introgressed during the soybean breeding and variety development.

### Example 16

### Epistatic Interactions Between the Rhg1 and Rhg4 Loci

Methods proceeded according to Examples 1-6, unless described otherwise.

It has been reported that the interaction of two or more alleles (epistasis) plays a major role in an organism's resistance to diseases and pests [Nagel; Bayless et al., 2016]. The Rhg1 GmSNAP18 protein interacts with NSF (N-ethylmaleimide-sensitive factor) protein and disturbs vesicle trafficking [Bayless et al., 2018; Bayless et al., 2016]. It is also well-documented that epistasis occurs in ‘Peking’-derived SCN resistance, in which the ‘Peking’-type Rhg1-a has high efficacy when the ‘Peking’-type Rhg4 is also present [Brucker et al.]. However, until now the genetic basis underlying high efficacy resistance was unknown. The present study shows that all the 106 soybean lines were grouped into six categories based on the genomic variation of Rhg1 and Rhg4 loci (FIG. 6). Among these, 11 lines carrying 4.7 to 9.4 copies of Rhg1 mainly showed resistance to races 3 and 4, while 12 lines carrying both the Peking'-type of Rhg1-a and Rhg4 (2.2-4.3 copies) showed greater resistance to races 1, 3 and 5 and were genotypically clustered. Importantly, PI 437654 exhibited high resistance to multiple SCN races, including races 1, 2, 3, 5 and 14 [Gardner et al.; Liu et al., 2017]. Our analysis has revealed that PI 437654 carries 3.3 copies of Peking'-type Rhgl-a and 4.3 copies of the Peking'-type Rhg4. Cultivar ‘Peking’ carries 3.2 copies of the Peking'-type Rhgl-a and 2.3 copies of ‘Peking’-type Rhg4. It is likely that the CNV of the Rhg4 gene impacts the different SCN resistance levels found between PI 437654 and ‘Peking’.

Interestingly, among SCN resistant PIs characterized in the present study, PI 407729, did not carry any known SCN resistance loci (Rhg4 or Rhg1) but still showed resistance to multiple SCN races. This can be explained, in part, by the presence of the SNP in the GmSHMT08⁺ promoter. These variations may correspond to trans-acting elements that can regulate other novel genes involved in SCN resistance beside classic Rhg1 and Rhg4 loci, and hence warrants further promoter analysis and gene functional characterization. Furthermore, genetic mapping of the PI 407729 resistant QTL may reveal a previously unknown SCN resistance locus, conferring a unique mode of resistance. Results obtained from the current study demonstrated that broad-based resistance to multiple SCN races requires very specific haplotypes of the Rhg1 and Rhg4 loci at the promoter, amino acid sequences and CNV. In fact, the type of interaction between the different alleles confers resistance to a given race that is haplotype-dependent. This study shows that having more copies of GmSHMT08 provides more transcript abundance, therefore reinforcing the resistance to SCN. Similar observations have been also revealed in the case of the GmSNAP18 gene.

The genetic basis for broad-based resistance to multiple races elucidated in the present study will greatly benefit soybean breeders in the development of SCN-resistance varieties. In addition, it will also help to select parental lines to design future crosses and trait introgressions. The SNP marker assays associated with CNV and SNP/indels can be used to stack multi-copy of the Rhgl-b (PI88788-type of resistance) or Rhg4 (‘Peking’ type resistance) for breeding purposes and it will provide more sources for broad-spectrum SCN resistance.

In summary, results obtained from the Examples reveal several new discoveries. (1) The Rhg4 locus is a highly repeated region similar to the Rhg1 locus, likely consisting of a 35.7-kb tandem repeat unit. Eleven lines with resistance to multiple races of SCN exhibited a CNV of 2.1 to 4.3 copies of Rhg4 coupled with a ‘Peking’-type Rhgl-a with copy numbers ranging from 1.9 to 3.5. (2) The lines with PI 88788-type Rhgl-b haplotypes required greater than 5.6 copies to confer resistance to SCN races 3 and 14, regardless of the Rhg4 haplotype. (3) When GmSNAP18 copy number dropped below 5.6 copies, a Peking type GmSHMT08 haplotype was required to ensure resistance to SCN pointing to a novel mechanism of epistasis between the GmSNAP18 and GmSHMT08 involving minimum requirements for copy numbers at both loci. (4) ‘Cloud’-type Rhg1 performed better than ‘PI 88788’-type Rhg1 and required less GmSNAP18 copy numbers to confer SCN resistance. (5) When soybean lines cumulated more copies of the GmSHMT08 gene, they acquired broad resistance to SCN. (6) Soybean lines with low CNV (1 to 3 copies) of Peking'-type Rhgl-a but lacking Rhg4 allele showed resistance only to SCN race 5. (7) Both Rhg1 and Rhg4 loci were in strong LD with the surrounding regions of the genome. (8) Expression analysis showed that transcript abundance of the GmSHMT08 in root tissue correlates with more copies of the Rhg4 locus, reinforcing the resistance to SCN. (9) Haplotype analysis of the GmSHMT08 and GmSNAP18 promoters provide an additional layer of the resistance mechanism. These findings provide new insight into epistatsis, haplotype compatibility, Copy Number Variants, promoter variation, and its impact on broad-based disease resistance.

### Example 17

### Functional Analysis of the GmSHMT08 Promoter (Transgenic Soybean Root) and Discovery of the MADS SQUAMOSA-box Transcription Factor Binding Site and its Role in SCN Susceptibility/Resistance

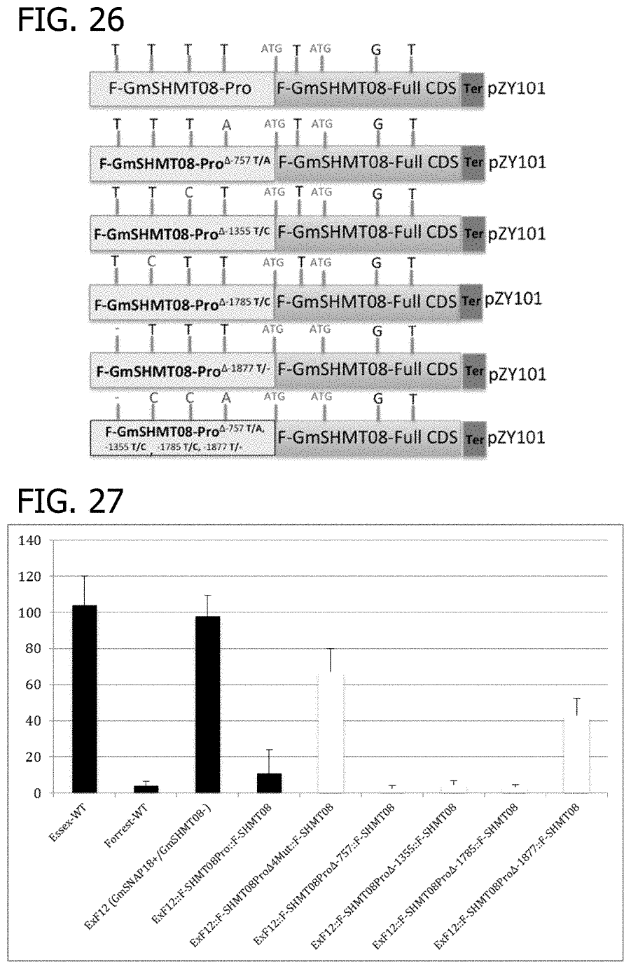

Functional analysis was performed on the GmSHMT08 promoter carrying the four SNPs at four positions within the 2 Kb promoter F-GmSHMT08-Pro^{Δ-757 TIA}, F-GmSHMT08-Pro^{Δ-1355 T/C}, F-GmSHMT08-Pro^{Δ-1785 T/C}, F-GmSHMT08-Pro^{Δ-1877 T/-} independently. The F-GmSHMT08-Pro^{Δ-757 T/A}, Pro^{Δ-1355 T/C, -1785 T/C, -1877 T/-} construct carries all the four SNPs. Each construct contained the endogenous GmSHMT08 promoter, in addition to the GmSHMT08 coding sequence as shown in FIG. 26.

A ExF12 (Essex x Forrest) RIL line carrying the resistant GmSNAP1⁺ allele but the susceptible GmSHMT08⁻ allele [Lakhssassi et al.] has been used for the soybean composite root transformation.

ExF12 presented 97 cysts on average; therefore, it was completely susceptible to SCN.

As expected, susceptible ExF12 transgenic hairy root carrying the F-GmSHMT08-Pro::GmSHMT08-CDS (positive control) decreased the number of SCN cysts to nearly 11 in transgenic soybean roots. Both the GmSHMT08-WT endogenous promoter and the GmSHMT08-WT CDS responded to SCN infections and the ExF12 line become resistant to SCN.

Interestingly, when the construct carried the four susceptible SNPs at the Forrest GmSHMT08-Pro; F-GmSHMT08-Pro^{Δ-757 T/A, -1355 T/C, -1785 T/C, -1877 T/-}, the screened transgenic ExF12 lines presented 67 cysts in average, and therefore was susceptible to SCN. This suggests that at least one, two, three or the four SNPs tested on the F-GmSHMT08-Pro may be responsible for the observed susceptibility.

When tested independently, transgenic ExF12 lines' expressing the following independent constructs: F-GmSHMT08-Pro^{Δ-757 T/A}, F-GmSHMT08-Pro^{Δ-1355 T/C}, F-GmSHMT08-Pro^{Δ-1785 T/C} showed decreased in cyst number with 2, 4, and 3 cysts in average, respectively.

Interestingly, transgenic ExF12 lines expressing the F-GmSHMT08-Pro^{Δ-1877 T/-} construct presented 42 cysts on average, and therefore was susceptible to SCN. This directly points to the role of the SNP at position −1877 T/- (corresponding to the loss of the MADS SQUAMOSA-box TFBS) in SCN susceptibility/resistance.

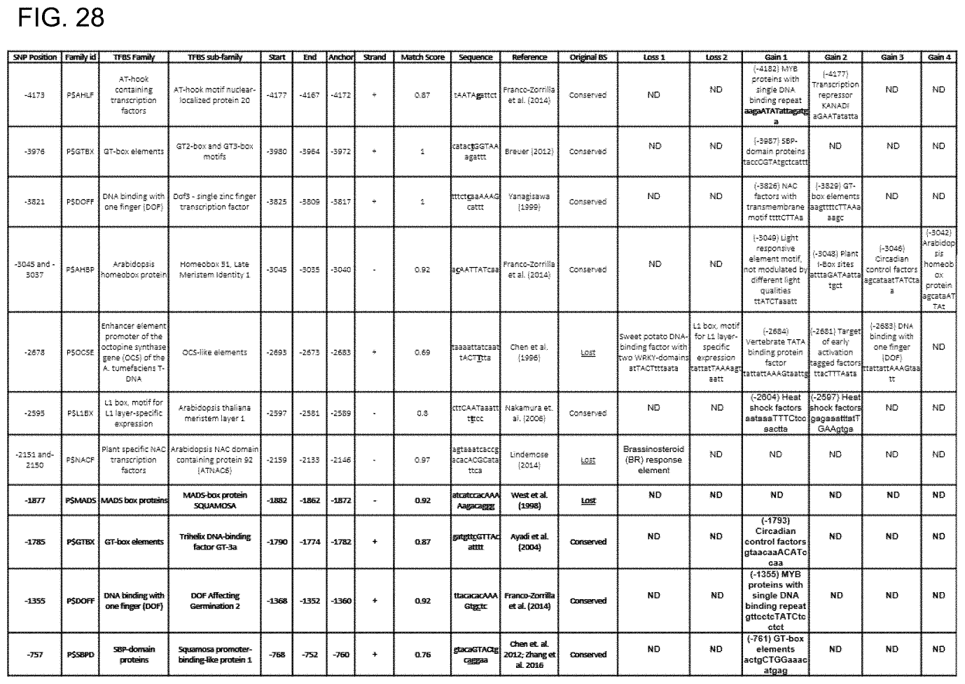

Full data on cyst number present in tested lines with various GmSHMT08 promoter mutations is shown in FIG. 27. Furthermore, in silico analysis of the GmSHMT08 promoter is shown in FIG. 19B and FIG. 28.

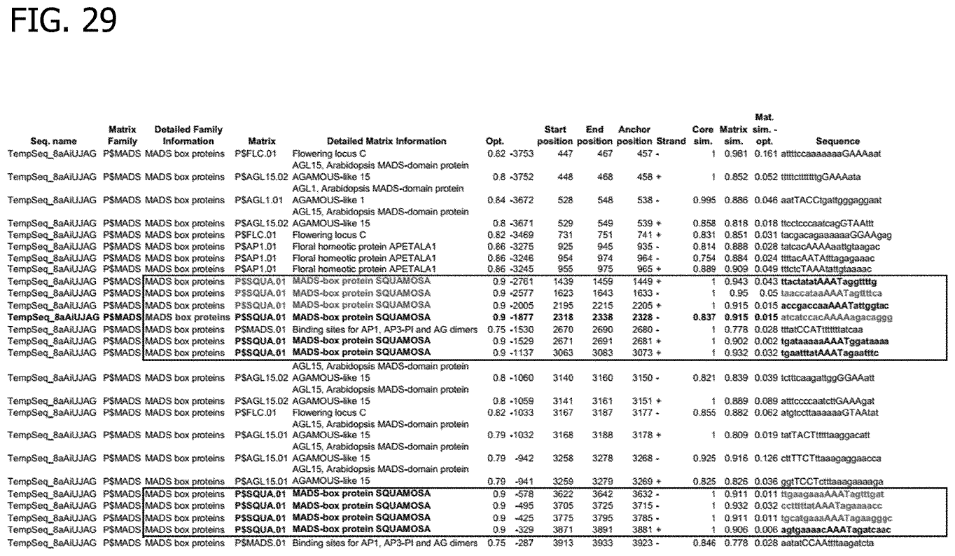

In total, 10 MADS SQUAMOSA-box Transcription Factor Binding Sites (TFBS) are present at the GmSHMT08 promoter of soybean susceptible lines. Five MADS SQUAMOSA-box were on the positive (+) strand, and the other five were present on the negative (−) strand (see FIG. 29). Most of the MADS SQUAMOSA-box TFBS recognizes the following sequence: AAAT. However, only one out of the 10 MADS SQUAMOSA-box TFBS (at position −1877 in the Figure bellow) presented a different binding sequence; AAAA, on the susceptible soybean lines. Because of the INDEL at position −1877 (-/T), the resistant lines lost this “special” MADS SQUAMOSA-box TFBS (AAAA).

Five MADS SQUAMOSA-box were on the positive (+) strand.

1 > + 2761
ttactatatAAATaggttttg
2 > + 2005
accgaccaaAAATattggtac
3 > + 1529
tgataaaaaAAATggataaaa
4 > + 1137
tgaatttatAAATagaatttc
5 > + 329
agtgaaaacAAATagatcaac
TGATAAAAAAAATGGATAAAA
AGTGAAAACAAATAGATCAAC
ACCGACCAAAAATATTGGTAC
TTACTATATAAATAGGTTTTG
TGAATTTATAAATAGAATTTC

Five MADS SQUAMOSA-box were on the negative (−) strand.

```
6 > − 2577
taaccataaAAATagttttca
7 > − 1877
atcatccacAAAAagacaggg
8 > − 578
ttgaagaaaAAATagtttgat
9 > − 495
cctttttatAAATagaaaacc
10 > − 329
tgcatgaaaAAATagaagggc
−−CCTTTTTATAAATAGAAAACC
−−TGCATGAAAAAATAGAAGGGC
  −TAACCATAAAAATAGTTTTCA
    ATCATCCACAAAAAGACAGGG−−
−−TTGAAGAAAAAATAGTTTGAT
```

Within the 2 Kb GmSHMT08 promoter, the INDEL at position −1877 T/- was the only SNP that resulted to the loss of the MADS SQUAMOSA-box TFBS in resistant lines. All the other observed SNPs did not impact the presence of their corresponding TFBS between SCN resistant and susceptible lines.

### REFERENCES

Aflitos S et al. Exploring genetic variation in the tomato (Solanum section Lycopersicon) clade by whole-genome sequencing. The Plant Journal (2014), 80: 136-148.

Aitman TcJ et al. Copy number polymorphism in Fcgr3 predisposes to glomerulonephritis in rats and humans. Nature (2006), 439: 851-855.

Albertini AcM et al. On the formation of spontaneous deletions: The importance of short sequence homologies in the generation of large deletions. Cell (1982), 29: 319-328.

Anderson JcE et al. A roadmap for functional structural variants in the soybean genome. G3: Genes, Genomes, Genetics (2014), 4: 1307-1318.

Arelli AcP et al. Soybean germplasm resistant to Races 1 and 2 of Heterodera glycines. Crop Science (1997), 37: 1367-1369.

Arelli PcR et al. Soybean reaction to Races 1 and 2 of Heterodera glycines. Crop Science (2000), 40: 824-826.

Arelli PcR et al. Inheritance of resistance in soybean PI 567516C to LY1 nematode population infecting cv. Hartwig. Euphytica (2009), 165: 1-4.

Ausubel et al. Short Protocols in Molecular Biology, 5th ed., Current Protocols, 2002.

Bayless A M et al. Disease resistance through impairment of α-SNAP-NSF interaction and vesicular trafficking by soybean Rhgl. Proceedings of the National Academy of Sciences (2016), 113: E7375-E7382.

Bayless A M et al. An atypical N-ethylmaleimide sensitive factor enables the viability of nematode-resistant Rhg1 soybeans. Proceedings of the National Academy of Sciences (2018), 115: E4512-E4521.

Brown S et al. A high-throughput automated technique for counting females of Heterodera glycines using a fluorescence-based imaging system. Journal of Nematology (2010), 42: 201-206.

Brucker E et al. Rhg1 alleles from soybean PI 437654 and PI 88788 respond differentially to isolates of Heterodera glycines in the greenhouse. Theoretical and Applied Genetics (2005), 111: 44-49.

Choi J-W et al. Whole-genome resequencing analyses of five pig breeds, including Korean wild and native, and three European origin breeds. DNA Research (2015), 22: 259-267.

Concibido V C et al. A decade of QTL mapping for cyst nematode resistance in soybean. Crop Science (2004), 44: 1121-1131.

Cook D E et al. Copy number variation of multiple genes at Rhg1 mediates nematode resistance in soybean. Science (2012), 338: 1206-1209.

Cook D E et al. Distinct copy number, coding sequence, and locus methylation patterns underlie Rhgl-mediated soybean resistance to soybean cyst nematode. Plant Physiology (2014), 165: 630-647.

Dassanayake M et al. The genome of the extremophile crucifer Thellungiella parvula. Nature Genetics (2011), 43: 913-918.

de Koning A P et al. Repetitive elements may comprise over two-thirds of the human genome. PLoS Genetics (2011), 7: e1002384.

DeBolt S. Copy number variation shapes genome diversity in Arabidopsis over immediate family generational scales. Genome Biology and Evolution (2010), 2: 441-453.

Dobbels A A et al. An induced chromosomal translocation in soybean disrupts a KASI ortholog and is associated with a high-sucrose and low-oil seed phenotype. G3: Genes, Genomes, Genetics (2017), 7: 1215-1223.

Elhai and Wolk. Conjugal Transfer of DNA to Cyanobacteria. Methods in Enzymology (1988), 167: 747-754.

Flagel L E & Wendel J F. Gene duplication and evolutionary novelty in plants. New Phytologist (2009), 183: 557-564.

Gardner M et al. Genetics and adaptation of soybean cyst nematode to broad spectrum soybean resistance. G3: Genes, Genomes, Genetics (2017), 7: 835-841.

Gibbs R A et al. The international HapMap project. Nature (2003), 426: 789-796.

Gore MA et al. A first-generation haplotype map of maize. Science (2009), 326: 1115-1117.

Heinberg A et al. Direct evidence for the adaptive role of copy number variation on antifolate susceptibility in Plasmodium falciparum. Molecular Microbiology (2013), 88: 702-712.

Huang X et al. Resequencing rice genomes: an emerging new era of rice genomics. Trends in Genetics (2013), 29: 225-232.

Inoue K & Lupski J R. Molecular mechanisms for genomic disorders. Annual Review of Genomics and Human Genetics (2002), 3: 199-242.

Jackson S A et al. Sequencing crop genomes: approaches and applications. New Phytologist (2011), 191: 915-925.

Kadam S et al. Genomic-assisted phylogenetic analysis and marker development for next generation soybean cyst nematode resistance breeding. Plant Science (2016), 242: 342-350.

Kandoth P K et al. Systematic mutagenesis of serine hydroxymethyltransferase reveals essential role in nematode resistance. Plant Physiology (2017), 175: 1370-1380.

Karthikraja V et al. Types of interfaces for homodimer folding and binding. Bioinformation (2009), 4: 101-111.

Lakhssassi N et al. Characterization of the soluble NSF attachment protein gene family identifies two members involved in additive resistance to a plant pathogen. Scientific Reports (2017), 7: 45226.

Lam H M et al. Resequencing of 31 wild and cultivated soybean genomes identifies patterns of genetic diversity and selection. Nature Genetics (2010), 42: 1053-1059.

Lam H M et al. Addendum: Resequencing of 31 wild and cultivated soybean genomes identifies patterns of genetic diversity and selection. Nature Genetics (2011), 43: 387-387.

Lee T G et al. Evolution and selection of Rhgl, a copy-number variant nematode-resistance locus. Molecular Ecology (2015), 24: 1774-1791.

Li Y H et al. De novo assembly of soybean wild relatives for pan-genome analysis of diversity and agronomic traits. Nature Biotechnology (2014), 32: 1045-1052.

Lim U et al. Polymorphisms in cytoplasmic serine hydroxymethyltransferase and methylenetetrahydrofolate reductase affect the risk of cardiovascular disease in men. Journal of Nutrition (2005), 135: 1989-1994.

Liu S M et al. A soybean cyst nematode resistance gene points to a new mechanism of plant resistance to pathogens. Nature (2012), 492: 256-260.

Liu S et al. The soybean GmSNAP18 gene underlies two types of resistance to soybean cyst nematode. Nature Communications (2017), 8: 14822.

Macdonald M A et al. A novel gene containing a trinucleotide repeat that is expanded and unstable on Huntington's disease chromosomes. The Huntington's Disease Collaborative Research Group. Cell (1993), 72: 971-983.

Maddocks O D et al. Serine metabolism supports the methionine cycle and DNA/RNA methylation through de novo ATP synthesis in cancer cells. Molecular Cell (2016), 61: 210-221.

Matthews B F et al. Engineered resistance and hypersusceptibility through functional metabolic studies of 100 genes in soybean to its major pathogen, the soybean cyst nematode. Planta (2013), 237: 1337-1357.

McHale L K et al. Structural variants in the soybean genome localize to clusters of biotic stress-response genes. Plant Physiology (2012), 159: 1295-1308.

McKenna A et al. The genome analysis toolkit: a MapReduce framework for analyzing next-generation DNA sequencing data. Genome Research (2010), 20: 1297-1303.

Milne I et al. Flapjack-graphical genotype visualization. Bioinformatics (2010), 26: 3133-3134.

Moore R C & Purugganan M D. The evolutionary dynamics of plant duplicate genes. Current Opinion in Plant Biology (2005), 8: 122-128.

Myers R. Huntington's disease genetics. NeuroRx (2004), 1: 255-262.

Nagel R L. Epistasis and the genetics of human diseases. Comptes Rendus Biologies (2005), 328: 606-615.

Niblack T L et al. Soybean cyst nematode in Illinois from 1990 to 2006: Shift in virulence phenotype of field populations. Journal of Nematology (2006), 38: 285-285.

Olsen K M & Wendel J F. A bountiful harvest: genomic insights into crop domestication phenotypes. Annual Review of Plant Biology (2013), 64: 47-70.

Paone A et al. SHMT1 knockdown induces apoptosis in lung cancer cells by causing uracil misincorporation. Cell Death & Disease (2014), 5: e1525.

Patil G et al. Soybean (Glycine max) SWEET gene family: insights through comparative genomics, transcriptome profiling and whole genome re-sequence analysis. BMC Genomics (2015), 16:520.

Patil G et al. Genomic-assisted haplotype analysis and the development of high-throughput SNP markers for salinity tolerance in soybean. Scientific Reports (2016), 6: 19199.

Perry G H et al. Diet and the evolution of human amylase gene copy number variation. Nature Genetics (2007), 39: 1256-1260.

Perry G H et al. Copy number variation and evolution in humans and chimpanzees. Genome Research (2008), 18: 1698-1710.

Qi X P et al. Identification of a novel salt tolerance gene in wild soybean by whole-genome sequencing. Nature Communications (2014), 5: 4340.

Rambani A et al. The methylome of soybean roots during the compatible interaction with the soybean cyst nematode, Heterodera glycines. Plant Physiology (2015), 168: 1364-1377.

Redon R et al. Global variation in copy number in the human genome. Nature (2006), 444: 444-454.

Rubin C-J et al. Whole-genome resequencing reveals loci under selection during chicken domestication. Nature (2010), 464: 587-591.

Sambrook and Russel. Molecular Cloning: A Laboratory Manual, 3rd ed. Cold Spring Harbor Laboratory Press, 2001.

Sambrook and Russel. Condensed Protocols from Molecular Cloning: A Laboratory Manual. Cold Spring Harbor Laboratory Press, 2006.

Schmutz J et al. Genome sequence of the palaeopolyploid soybean. Nature (2010), 465: 120-120. [Corrigendum of Schmutz J et al. Genome sequence of the palaeopolyploid soybean. Nature (2010), 463: 178-183.]

Schmutz J et al. A reference genome for common bean and genome-wide analysis of dual domestications. Nature Genetics (2014), 46: 707-713.

Sebat J et al. Large-scale copy number polymorphism in the human genome. Science (2004), 305: 525-528.

Sharp A J et al. Segmental duplications and copy-number variation in the human genome. American Journal of Human Genetics (2005), 77: 78-88.

Shlien A & Malkin D. Copy number variations and cancer. Genome Medicine (2009), 1: 62.

Skibola C F et al. Polymorphisms in the thymidylate synthase and serine hydroxymethyltransferase genes and risk of adult acute lymphocytic leukemia. Blood (2002), 99: 3786-3791.

Song Q et al. Construction of high resolution genetic linkage maps to improve the soybean genome sequence assembly Glyma1.01. BMC Genomics (2016), 17: 33.

Song Q et al. Genetic characterization of the soybean nested association mapping population. The Plant Genome (2017), 10: 10.3835.

Telenti A et al. Deep sequencing of 10,000 human genomes. Proceedings of the National Academy of Sciences (2016), 113: 11901-11906.

Valliyodan B et al. Landscape of genomic diversity and trait discovery in soybean. Scientific Reports (2016), 6: 23598.

Varshney R K et al. Whole-genome resequencing of 292 pigeonpea accessions identifies genomic regions associated with domestication and agronomic traits. Nature Genetics (2017), 49: 1082-1088.

Varshney R K et al. Draft genome sequence of chickpea (Cicer arietinum) provides a resource for trait improvement. Nature Biotechnology (2013), 31: 240-246.

Vuong T D et al. Novel quantitative trait loci for broad-based resistance to soybean cyst nematode (Heterodera glycines Ichinohe) in soybean PI 567516C. Theoretical and Applied Genetics (2010), 121: 1253-1266.

Wan Jet al. Application of Digital PCR in the Analysis of Transgenic Soybean Plants. Advances in Bioscience and Biotechnology (2016), 7: 403-417.

Wang L H et al. Genome sequencing of the high oil crop sesame provides insight into oil biosynthesis. Genome Biology (2014), 15: R39.

Wrather J A & Koenning S R. Estimates of disease effects on soybean yields in the United States 2003 to 2005. Journal of Nematology (2006), 38: 173-180.

Wu X et al. Q T L, additive and epistatic effects for SCN resistance in PI 437654. Theoretical and Applied Genetics (2009), 118: 1093-1105.

Xu X et al. Resequencing 50 accessions of cultivated and wild rice yields markers for identifying agronomically important genes. Nature Biotechnology (2012), 30: 105-111.

Yano K et al. Genome-wide association study using whole-genome sequencing rapidly identifies new genes influencing agronomic traits in rice. Nature Genetics (2016), 48: 927-934.

Zarrei M et al. A copy number variation map of the human genome. Nature Reviews Genetics (2015), 16: 172-183.

Zhou Z K et al. Resequencing 302 wild and cultivated accessions identifies genes related to domestication and improvement in soybean. Nature Biotechnology (2015), 33: 408-414.

Zhou X et al. Population genomics reveals low genetic diversity and adaptation to hypoxia in snub-nosed monkeys. Molecular Biology and Evolution (2016), 33: 2670-2681.

What is claimed is:

1. A plant of an agronomically elite soybean variety, comprising a first polynucleotide encoding a serine hydroxymethyltransferase promoter that functions in the soybean plant operably linked to a second polynucleotide encoding a polypeptide having serine hydroxymethyltransferase activity;
wherein said first polynucleotide comprises SEQ ID NO: 1, or a sequence at least 95% identical thereto, or a full-length complement thereof, or a functional fragment thereof;
wherein said first polynucleotide further comprises one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-; and
wherein the plant has increased soybean cyst nematode (SCN) resistance compared to a control soybean plant lacking said first polynucleotide.

2. The plant of claim 1, wherein said polypeptide having serine hydroxymethyltransferase activity comprises SEQ ID NO: 2, or a sequence at least 95% identical thereto, or a full-length complement thereof, or a functional fragment thereof, and wherein said polypeptide having serine hydroxymethyltransferase activity further comprises one or more mutations of SEQ ID NO: 2 selected from the group consisting of: I107F, P200R, P200-, N459Y, and N459H.

3. The plant of claim 2, wherein said second polynucleotide has increased expression, an altered expression pattern, or an increased copy number.

4. The plant of claim 3, wherein said second polynucleotide has a copy number of at least 2.

5. The plant of claim 1, further comprising a third polynucleotide encoding an alpha soluble NSF attachment protein promoter that functions in the soybean plant operably linked to a fourth polynucleotide encoding a polypeptide having alpha soluble NSF attachment protein activity;
wherein said third polynucleotide comprises SEQ ID NO: 3, or a sequence at least 95% identical thereto, or a full-length complement thereof, or a functional fragment thereof; and
wherein said third polynucleotide further comprises one or more mutations of SEQ ID NO: 3 selected from the group consisting of: C1161A, C1082A, C1044A, C1025T, A1016C, T997A, C970A, C970-, G829T, G825T, A815C, A363T, T336C, G334A, T328C, T327A, C267G, T157G, T83A, C57T, and T36A.

6. The plant of claim 5, wherein said polypeptide having alpha soluble NSF attachment protein activity comprises SEQ ID NO: 4, or a sequence at least 95% identical thereto, or a full-length complement thereof, or a functional fragment thereof, and wherein said polypeptide having alpha soluble NSF attachment protein activity further comprises one or more mutations of SEQ ID NO: 4 selected from the group consisting of: A111D, Q203K, D208E, I238V, E285Q, D286Y, D286H, D287E, +287A, +287V, L288I, and +288T.

7. The plant of claim 6, wherein said fourth polynucleotide has increased expression, an altered expression pattern, or an increased copy number.

8. The plant of claim 7, wherein said fourth polynucleotide has a copy number of at least 2.

9. The plant of claim 3, further comprising a third polynucleotide encoding an alpha soluble NSF attachment protein promoter that functions in the soybean plant operably linked to a fourth polynucleotide encoding a polypeptide having alpha soluble NSF attachment protein activity;
wherein said third polynucleotide comprises SEQ ID NO: 3, or a sequence at least 95% identical thereto, or a full-length complement thereof, or a functional fragment thereof; and
wherein said third polynucleotide further comprises one or more mutations of SEQ ID NO: 3 selected from the group consisting of: C1161A, C1082A, C1044A, C1025T, A1016C, T997A, C970A, C970-, G829T, G825T, A815C, A363T, T336C, G334A, T328C, T327A, C267G, T157G, T83A, C57T, and T36A.

10. The plant of claim 9, wherein said polypeptide having alpha soluble NSF attachment protein activity comprises SEQ ID NO: 4, or a sequence at least 95% identical thereto, or a full-length complement thereof, or a functional fragment thereof, and wherein said polypeptide having alpha soluble NSF attachment protein activity further comprises one or more mutations of SEQ ID NO: 4 selected from the group consisting of: A111D, Q203K, D208E, I238V, E285Q, D286Y, D286H, D287E, +287A, +287V, L288I, and +288T.

11. The plant of claim 10, wherein said fourth polynucleotide has increased expression, an altered expression pattern, or an increased copy number.

12. The plant of claim 11, wherein said fourth polynucleotide has a copy number of at least 2.

13. A plant part of the plant of claim 1.

14. A plant of an agronomically elite soybean variety, comprising a first polynucleotide encoding a serine hydroxymethyltransferase promoter that functions in the soybean plant operably linked to a second polynucleotide encoding a polypeptide having serine hydroxymethyltransferase activity;
wherein said polypeptide having serine hydroxymethyltransferase activity comprises SEQ ID NO: 2, or a sequence at least 95% identical thereto, or a full-length complement thereof, or a functional fragment thereof;
wherein said polypeptide having serine hydroxymethyltransferase activity further comprises one or more mutations of SEQ ID NO: 2 selected from the group consisting of: I107F, P200R, P200-, N459Y, and N459H;
wherein the plant has increased soybean cyst nematode (SCN) resistance compared to a control soybean plant lacking said second polynucleotide; and
wherein said second polynucleotide has increased expression, an altered expression pattern, or an increased copy number.

15. The plant of claim 14, wherein said second polynucleotide has a copy number of at least 2.

16. The plant of claim 14, further comprising a third polynucleotide encoding an alpha soluble NSF attachment protein promoter that functions in soybean operably linked to a fourth polynucleotide encoding a polypeptide having alpha soluble NSF attachment protein activity;
wherein said third polynucleotide comprises SEQ ID NO: 3, or a sequence at least 95% identical thereto, or a full-length complement thereof, or a functional fragment thereof; and
wherein said third polynucleotide further comprises one or more mutations of SEQ ID NO: 3 selected from the group consisting of: C1161A, C1082A, C1044A, C1025T, A1016C, T997A, C970A, C970-, G829T, G825T, A815C, A363T, T336C, G334A, T328C, T327A, C267G, T157G, T83A, C57T, and T36A.

17. The plant of claim 16, wherein said polypeptide having alpha soluble NSF attachment protein activity comprises SEQ ID NO: 4, or a sequence at least 95% identical thereto, or a full-length complement thereof, or a functional fragment thereof, and wherein said polypeptide having alpha soluble NSH attachment protein activity further comprises one or more mutations of SEQ ID NO: 4 selected from the group consisting of: A111D, Q203K, D208E, I238V, E285Q, D286Y, D286H, D287E, +287A, +287V, L288I, and +288T.

18. The plant of claim 17, wherein said fourth polynucleotide has increased expression, an altered expression pattern, or an increased copy number.

19. The plant of claim 18, wherein said fourth polynucleotide has a copy number of at least 2.

20. A plant part of the plant of claim 14.

21. A DNA construct comprising a first polynucleotide encoding a serine hydroxymethyltransferase promoter that functions in a soybean plant operably linked to a second polynucleotide encoding a polypeptide having serine hydroxymethyltransferase activity;
wherein said first polynucleotide comprises SEQ ID NO: 1, or a sequence at least 95% identical thereto, or a full-length complement thereof, or a functional fragment thereof; and
wherein said first polynucleotide further comprises one or more mutations of SEQ ID NO: 1 selected from the group consisting of: A3959T, G3726C, A3444T, C3147T, A3130C, T3037C, G2999C, C2998T, T2979C, C2846T, G2475T, A2420G, C2416T, +2323T, T2051A, G2050C, A1606G, T1523-, G1164A, T1156A, A403C, C380T, A338T, T329A, T313C, T225G, T225-, A133G, A133-, G28T, and G28-.


SEQUENCE LISTING (4 sequences)

SEQ ID NO: 1 - DNA, 4200 bp, Glycine max
tcatgccactaaaactatcatctaatagattctttgacatctaaggactaattgaataaatacaattaagtaaaattgtctatgatttaggcctgtggaataatccttgagtaagcctttattgacatcgctaacaagtagcatgtcattaaggtttcattcgatggtattgatcaggcctctataaaattttgtacattttaatatgcatcaaatgagcatactggtaaagatttcggtgctcaagttaatagttggtaaagtaaaagcattatatgtaagattttcatgtacttggtaaagctaagggactatcggagattgttgataagcatttaaaaaactctcaacaatcttctatctgcctataaagttttctcaaaaagcatttaaaaaatttataggttaattagagatttgttaggtaggttaacatacatgtaaagatttttctttttttggaaaatacatgtaaagagttttgtaaaagtagaacttgtgaatacgtgatttataagacaattcatattcctcccaatcaggtaattttgtgcaaaaagtcttattaagttggtgtgtaactgtatttaaaataaattcttcattgatggagtgggtgaagttcacgacatcgatcatagacagattttttttcttccattctaatgtttaccgtgtgattgtggccacaatcaatttgtaaacctgtgacaaactttgtctcttggcctacgacagaaaaaaggaagagctgaatgctgatggttgttccatagccactaaagtgagcatttgtagtacccatgtcttttggcactgtatgtgactcagctagttgcgctggatgggttccattccattgatttttttttttttatgttaaaattttcatttagtattctaaactcacttgacattaattttgtcttacaattttttgtgatatcaaccaagtttctctaaatattgtaaaacacaaaatatttatattccaatcttcaatgtttttatttgacattataaatatttaaaggatagaatcaatgttaatcaagttaacataaaaaataaaaaattacatagcattcaacatgtaggtatcaaatctatgttataaaatgtttattagatagagaaaaatatttgctaaaattttgataattgtgctatgtttatatgttgaatgatgggtaaaataaaatgacgcataattaagtaacataagtaaaataaaaattaagtttaatttttatgaattatcaatataaaaaaataaaatatattcctaacatttctctttcctctattttacattcattttattttcttaatttttttcattttgatatcctttaatataataactaatactgtaaaaaaaatgtcattttttattcgtaaacaacccggtcaaaatataggtttaacaattagtcaattactatataaataggttttgtatttgaatatgttagtaaaaagtagttttaatatatcttattccagtaaaattatcaattacttttaataataaagtcatacaaatttgtataaaactattttccccctacgataaaagttgtttcgaaaaaaaagtaagttggaaaaatttattgaagtgatgaaaactatttttatggttattttttatcacacaaattaattttggaatcttataattagaaatggttgaatttatatattggttaactttattttcttatttcgtccacagtaatgaattgtttcaaacaaaaaaaaaatcaattaatatatattttataattttactattgaaaaatacctttttcaaacaaatcactaatcacttaaaatacaaaaatatattttgatgagtgaaatatgactgcagaagtcaagtcccaaaattttgatgagtactactacaagggagatggggtcatcatagttgcttctgatttgctattttcattggtgtatgacctattaactattaactacgttgtggattgccaaatgaaaatatcagtgaagcaaaaccctcctcctccagcgctatgaatatgcgtgtgtcggtgatttacttgtacattttttgaaaatgaaagaaaacacaagcaaagtgaatgtgcatgtgatcaaataaaggaaacaaaccagcatacataaataatcaagctctggtccccagtcatggagattaatttgatgaaccgaccaaaaatattggtacagcataatcacaattattgagaagatatttttattttatttttaccgaatcgtcgcacgactcggcgtgttgcaaccgcattaaatctttgtgttggtctcaccctgtctttttgtggatgatcgatcctcttggattggtttttataaaactcaacttcccatcggtgttctttagtaattggagtatctttggatgttcgttacattttatgataaatttaaatgatccacaatcactaactcaattttgcaaagcaggattctgaatgtttttgtaaatctcgttttgtcctaaaagttcgtctataacaataaaacaaacatgcacttggttgtttttaaaattgtctcaaaactctgttataaagaaataagacctaaagatattttttacaaattttaattcaaacatgcactgatacatagagatatccttagattaatttattttgttgataaaaaaaatggataaaaatttccatgctttaaatttgtcattggtccatctgatcgactctatacatcaaacttgagtgttatttgcatacaaaaggaaaacatcagagacatgacagagtaggttgcattggtgtttagttgacctgattaagaagttacacacaaagtgctcctctatctcctcttcaaggtcctcctacctatagtcttcttgtacctcttattatatggattaattagtgtagaattatttcaacttaattaataattttgaatttaagtcatgagaatgagtatcaaaattttttcacctataaaaatcgaatgtgcttcaaataagattgtctctaataaataatatgtgtttaatctatattatttttatctgaatttataaatagaatttcagtattttttaaagatgaaattatcataattaattaaaagatatttatgttaattaaatttccccaatcttgaaagatattttatattacttttttaaggacattttgtattagctttgcgttaccagcttgctgcataagtacagctcaaggctttgaaaaacctatgtcggttggttcctctttaaagaaaagagaataaaaataacaaagaaaaaaaagtcgccttccatttcattcgcattcatagtaaaagagtgagcgatcccgggaaatgaattaatatacgactaaaaagatttgagaattataataattaataattaataattctttttcaaaagtaaagtacagtactgcaggaaacatgagcatgttcatagattaaaatttaaaagaatattatcagtaacaaaaaaataaaaattaacccatgcatccaagaaagaaatacccatgtgcttcagttgtccgctgtctgagatgtggtgaccttttttcaaatgatcataatagttacttcataatgacgacatgcatcaaactattttttcttcaaaaaatatattggcaccttttcactcggataattgcataaattattgttttaacttttatttgggttttctatttataaaaagggagatttttttaagaaaaaaaaaatcaaatcataggactcagatgactcgcccttctattttttcatgcaaatggccgttccaactattattactaatatgtaattattgaagcaaaacaattcaatgaccaaattaatgttaaaagtgaaaacaaatagatcaacaaccttctcgcgtagcttgagtagatcttaaaattggatatttgctcaattaatacgcttataatatagtagtagtagcctagatctagatgcagtttgtcccgcgttgtaattaaataaaatatcacggaattattatgagagcattggtgagcatgacaatttcaccggaaaaaaaaaagtgagcataataaagtcataaattaaaaactacaggtaggtaattgaggacttataacttggaagttgggacgtgccaaccgcatataacatacgcgattgaaagactctttacaaatggctttggca

SEQ ID NO: 2 - protein, 572 aa, Glycine max
MAPMPNGRFKSSPTSTSEIMNYEIKMLPTFKNEHIRTHISIPPQQTQQHSLFSLLAFRSSLTLIHSFPPFMDPVSVWGNTPLATVDPEIHDLIEKEKRRQCRGIELIASENFTSFAVIEALGSALTNKYSEGMPGNRYYGGNEYIDQIENLCRSRALQAFHLDAQSWGVNVQPYSGSPANFAAYTAVLNPHDRIMGLDLPSGGHLTHGYYTSGGKKISATSIYFESLPYKVNSTTGYIDYDRLEEKALDFRPKLIICGGSAYPRDWDYKRFREVADKCGALLLCDMAHTSGLVAAQEVNSPFEYCDIVTTTTHKSLRGPRAGMIFYRKGPKPPKKGQPENAVYDFEDKINFAVFPSLQGGPHNHQIGALAVALKQAASPGFKAYAKQVKANAVALGKYLMGKGYSLVTGGTENHLVLWDLRPLGLTGNIYRIGSLPSGFDLLQMSINLTCSLCDCFAGNKVEKLCDLCNITVNKNAVFGDSSALAPGGVRIGAPAMTSRGLVEKDFEQIGEFLHRAVTLTLEIQKEHGKLLKDFNKGLVNNKAIEDLKADVEKFSALFDMPGFLVSEMKYKD

SEQ ID NO: 3 - DNA, 1439 bp, Glycine max
ttcaacatgagtatgataataataataataaaaaattgttgttttctatttttactccaacatggactgaaattcatatgaatttttttgaatagtctatttttttttatttaatttaatattcatatcaaagttatttcatactgaaaaaaatattaaatactagcattctattattaccatttggaggaatgattgaaagagtgttaaagtgcaccttttcagtcaacagttaaaaataaggcgtttaattcaattcaatattacaaagttaagttggctgtataataataacagtggtagtaagtagtagagtgaaagaaaaatttttttggtcaaaatatttaaatcaagactagaagatatgcaaatcagagattacattggatgatacggtcgaccaataaaaaataaaagaaaaaacataaattgggatgttcaaatactaataataataactctaaacaaacattaacacgtgagttttctttcccacgttgtaatcattttgaatttttaaaatgttatgacacaaataataagttaataataattataatttaacatttgaattgataaaagtgtttagttttattgtagattaaactaatctttcttcgagtaaaaataacattaaattcctacacaacaggtttatcagtttatagagtaataacactcttattcttaatcgttttcttttctggaagaaaaaataaatcttagtcttgttatttttttgagaatgtaaaatataccttaaaaaattcccttaaagtttgtataattttttggtatgtaaatatatttataaataaaaaaatgtttgcgaaaagtaatatttacataacaaacactatttacagaacattgatgaaattatttttagatatataattattaatacgaatatatgaatatgttattaaagtaatcaatagttatgttaaaactgatctgttgactagacagtttgtcaatttattttttattcacttaattgctatttttttctaggtttgttctttcgttaaaaaaccttgcattggaggaaggccaatgctagttataaaaatataaaccatgatttgaatataaaattatttttagtcgaaaaacaatgaattatgttgcaagtatcactattgaaaaaatgccaacggagcccaagaaggtgaggcccaaactgaaagcgtgaagcggcccaagactgagtgaggaaataaataattatccagaaaatcggaaatggacaatccttcttgttacgcaattctgaatttgcgggttttggatttggacttggtcgtcaacacagtctaattaatatctttttgctccttcgcttatgaatcttcttcttcttcttcttgttcctgcaacgcactgaattcgatcaatcaatccatcttcaattgctttgtttcgatcggaggaaa

SEQ ID NO: 4 - protein, 288 aa, Glycine max
MADQLSKGEEFEKKAEKKLSGWGLFGSKYEDAADLFDKAANCFKLAKSWDKAGATYLKLASCHLKLESKHEAAQAHVDAAHCYKKTNINESVSCLDRAVNLFCDIGRLSMAARYLKEIAELYEGEQNIEQALVYYEKSADFFQNEEVTTSANQCKQKVAQFAAQLEQYQKSIDIYEEIARQSLNNNLLKYGVKGHLLNAGICQLCKEDVVAITNALERYQELDPTFSGTREYRLLADIAAAIDEEDVAKFTDVVKEFDSMTPLDSWKTTLLLRVKEKLKAKELEEDDL